(19) United States
(12) Patent Application Publication
(10) Pub. No.: US 2022/0238232 A1
(43) Pub. Date: Jul. 28, 2022

(54) SYSTEM AND METHOD FOR MEDICAL IMAGING INFORMATICS PEER REVIEW SYSTEM

(71) Applicant: TERARECON, INC., Foster City, CA (US)

(72) Inventors: Jeffrey Sorenson, Sussex, WI (US); Tiecheng Zhao, Concord, MA (US); David W. MacCutcheon, Marshfield, MA (US); Jacob Ian Taylor, Cambridge, MA (US); Misha Herscu, Amherst, MA (US); Gael Kuhn, Montreuil (FR); Steven Rothenberg, Cambridge, MA (US)

(21) Appl. No.: 17/717,900

(22) Filed: Apr. 11, 2022

Related U.S. Application Data
(63) Continuation of application No. 15/688,688, filed on Aug. 28, 2017.
(60) Provisional application No. 62/380,831, filed on Aug. 29, 2016.
(60) Provisional application No. 62/453,951, filed on Feb. 2, 2017.

(51) Int. Cl. G16H 50/20; G06T 7/00; G06Q 10/06; G16H 30/40; G16H 30/20; G06V 10/40

(52) U.S. Cl. CPC G16H 50/20; G06T 7/0012; G06Q 10/06398; G06T 2207/30004; G16H 30/20; G06V 10/40; G16H 30/40

(57) ABSTRACT

Image processing engines can be utilized to inject studies into other commercial or independently-developed peer review systems which are designed to review the medical findings identified by a set of physicians. Image processing engines detect, confirm or verify findings by physicians or other engines, where the engines operate as peer reviewers. The engines can prospectively “learn” from the feedback when these images are reviewed by the physicians during diagnostic interpretation creating a closed-loop quality assurance process and fostering a community platform approach to engine development which is supported by the security, governance, access control, regulatory compliance and other features of the Peer Review System. Utilizing machine learning based on the data collected from peer review, the Peer Review System can adapt and improve its performance as well as the measured performance of the physicians using the system for diagnostic interpretation.

### RELATED APPLICATIONS

This application is a continuation of U.S. Utility application Ser. No. 15/688,688 which was filed on Aug. 28, 2017, which claims the benefit of U.S. Provisional Application No. 62/380,831 filed Aug. 29, 2016, and U.S. Provisional Application No. 62/453,951 filed Feb. 2, 2017. The entirety of each of which is hereby incorporated by reference fully in their entirety.

### FIELD OF THE INVENTION

Embodiments of the present invention relate generally to medical information processing systems. More particularly, embodiments of the invention relate to using discrete containerized automated image processing engines and statistical methods of quality assurance and iterative image processing engine training.

### BACKGROUND

Image processing engines must perform well on live streams of incoming clinical data and not just the image/content cohorts. These real-time clinical images and content sets that need interpretation are often imperfect. This can occur because there are patient scanning defects such as scanning protocol errors, patient movement, metal artifacts, obese patients, etc. It may also happen due to a lack of availability of prior imaging studies that are related to the current one, or missing clinical information or medical errors, etc.

### BRIEF DESCRIPTION OF THE DRAWINGS

Embodiments of the invention are illustrated by way of example and not limitation in the figures of the accompanying drawings in which like references indicate similar elements.

FIG. 1 is a block diagram illustrating a medical data peer review system according to one embodiment of the invention.

FIG. 2 is a block diagram illustrating an example of an image processing server according to one embodiment.

FIG. 3 is a flow diagram illustrating a processing flow of medical image processing according to one embodiment.

FIGS. 4A-4C are block diagrams illustrating examples of configurations of image processing engines according to certain embodiments.

FIG. 5 is a flow diagram illustrating a processing flow of medical image processing according to another embodiment.

FIG. 6A is a flow diagram illustrating a workflow of a peer review process according to one embodiment.

FIG. 6B is a block diagram illustrating a peer review system according to one embodiment.

FIG. 7 shows an example of a data structure storing tracking data according to one embodiment.

FIG. 8 shows an example of a report of findings according to one embodiment.

FIG. 9 shows an example of a data structure storing tracking data according to another embodiment.

FIGS. 10A and 10B show examples of a data structure storing tracking data according to some embodiments.

FIG. 11 shows an example of a data structure storing tracking data and reports according to one embodiment.

FIGS. 12A-12C show examples of access control lists according to some embodiments.

FIG. 13 is a flow diagram illustrating a process for image processing according to one embodiment.

FIG. 14 is a flow diagram illustrating a process for image processing according to another embodiment.

FIG. 15 is a flow diagram illustrating a process for image processing according to another embodiment.

FIG. 16 is a flow diagram illustrating a process for image processing according to another embodiment.

FIG. 17 is a block diagram of a data processing system, which may be used with one embodiment of the invention.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

According to one aspect of the invention, a locally-sited system and/or cloud-based platform is utilized to make it easy to anonymize studies, upload studies, register and access a new account, establish a community, specify a clinical advisory board and/or governance for the group, access tools to train and create machine learned algorithms/engines, upload or download algorithms/engines, access and run published algorithms/engines on studies and communicate the outcome/results such as number of uses, accuracy, and confidence level based on the confirmed or rejected findings. The system can have a framework for determining interpretation workflow best practices which incorporate machine learned algorithms, which is configurable based on an individual's beliefs or a group's beliefs, along with big data analyses to determine similarities and crowd-sourced common practices between them. Algorithms which are configurable based on an individual's beliefs or a group's beliefs can be shared to one or more medical institutions.

The system can be a local cloud system for one medical institute at one or more locations. The cloud-based system can be a private cloud for one medical institute at one or more geographic locations. The system can be a system that can connect one or more medical institutes at one or more locations. The cloud-based system can be a public cloud. There can be a main cloud system that can connect multiple local cloud systems. For example, there can be a main cloud system that can connect multiple private cloud systems from multiple institutes. The degree of access of information and tools from the main cloud system to the private cloud systems can depend on preconfigured settings by the medical institutes.

The image processing engines can work independently or in combination with one another when evoked or enabled on a multi-sided platform which utilizes machine learning, deep learning and deterministic statistical methods (engines) running on medical image data, medical metadata and other patient and procedure related content to achieve improved cohorts of images and information that have a higher or lower pre-test probability or confidence of having a specific targeted finding confirmed by the physician or clinician. The target findings are either held in blind confidence to see if the physician agrees independently, or the findings are presented within the physician interpretation process to evoke responses, and any feedback, adjustments, agreement or disagreement are captured and utilized as performance feedback for the engines which created the suggestions.

Findings represent any anatomy of interest, measurements, indications, reference materials which may relate, prior studies similar to this study, suggestion of tools, and almost any then-current information or automations tools that are desired to be used in a diagnostic or research interpretation process. The engines are interchangeable in the Peer Review System and are not the invention. As such, the types of findings and functions of the engines will vary and change over time. The performance feedback received from both engines processing data and Peer Review System utilization data is captured in the form of statistical interactions data, and is utilized to determine the relative value of the current image data cohort which was processed and reviewed, as well as the value and accuracy of any tool(s), automated interactions, findings and other data intentionally evoked in the process of preparing the studies or displaying the study for physician or clinician review, and/or the value of the engine(s) themselves as well as various combinations thereof.

As such, every intentional or incidental presentation of images, data and findings can be measured for physician agreement, adjustment or disagreement, in either a blinded or unblinded manner, and this generates valuable data allowing any or all of the following: a) engines to be improved when new feedback is returned to allow for higher confirmation rates and reduction of missed findings by physicians, b) workflow to be improved by measuring reviewer performance and adapting review tools and image/content display to reduce effort and access to the commonly required items within the medical image (or other) viewer c) physician quality to be measured when curated image cohorts with known findings are sent (or injected) for peer review and blinded findings are compared to the actual known findings in the cohort, and d) prospective application of the Peer Review System to pre-process studies which have not been read, allowing real-time physician first-time interpretations of medical image studies to prospectively incorporate parallel blinded or unblinded automatically generated Peer Review System findings and e) assembly of a machine and human readable database(s) of such images, data, interactions, and findings which is/are updated iteratively or continuously to provide the ability to assess trends and/or to have supervised or unsupervised engines analyze this data continuously or iteratively in order to optimize the engine(s) that are used to create the next image cohort, tools and layout selection/suggestions, optional engine availability and other features and data necessary for peer review and diagnostic interpretation of this cohort (engines of engines).

One embodiment of the invention allows for an unsupervised (or supervised) engine of engines (also referred to as a master engine, a supervisor engine, or a managing engine) to run autonomously and select the engines (e.g., secondary engines or slave engines) that run and the number of image studies or patient content sets that run per engine, for example concurrently (e.g., via multiple threads) in a distributed manner. To achieve an autonomous capability, the Peer Review System administrator is required to provide the engine of engines limitations on the number of studies or content sets that it places in a cohort, or that it sends for peer review, each limited by time period or by a limit of uses of an engine or engines in any cohort, limitations or targets for the type and quantity of findings, specifications regarding the group(s) of cohorts, or time period(s).

The unsupervised engine of engines is provided individual and/or collective limitations (minimum, maximum, mean, or other statistically relevant or set limits/targets) on the types and/or number/amount of images, image cohorts, content, findings, interactions and processing time to run these engines and/or for physicians to perform these processes in order to force the engine of engines to optimize its work and not consume too much of the physicians' time for peer review and also not to consume too much computational resources, both of which have significant costs. To ensure alignment of the observations and selections of the unsupervised engine with maximized clinical value of the findings, annotation adjustments, assembly of image cohorts and physician/clinician feedback received in peer review and clinical diagnostic interpretation, weighted values (equal or unequal) are placed upon one or more of the a) engines, b) the quantity and type of findings made by each engine (including no findings) c) multipliers applied to the cases where findings are confirmed or rejected by multiple engines, and d) multipliers applied to the cases where multiple engines worked on an image or content set to determine a finding (or non-finding).

Engines may be developed by the same or different individuals or companies that developed the Peer Review System, with the engines utilizing commonalities in their input and output schemas to allow multiple engines to be run in serial or hierarchical fashion, also known as an ensemble engine. These ensemble engines can be assembled programmatically, using a supervised engine of engines, or an unsupervised engine of engines with values placed on the outputs or the running of engines or finding of findings. A pre-defined input and output schema for communications by and between engines and the Peer Review System, or between engines and other engines, allows for abstraction of inputs and outputs into various forms as required by various engines. For example, if Engine 1 accepts data point coordinates with zero as the middle value of an infinite positive and negative range domain and Engine 2 accepts these with 0 being the lowest value in an infinite always positive range domain, then the abstraction occurring in the communication schema would be to map the range values of these two domains over a specified range of possible shared values. The implementation of the abstraction method to implement the operation of containerized engines and engines of engines, works across all possible value types and ranges.

Findings can include but are not limited to derived images, contours, segmentations, overlays, numbers, similarities, quantities and any other values commonly viewed, measured, derived or found in enterprise electronic health records systems, picture archiving and communications systems, content management systems, peer review systems, laboratory systems and/or advanced visualization systems. Differences between the peer review generated results and the physician or clinician generated results can be captured, compared and output by the Peer Review System for future analyses and optimization of engines.

As a multi-tenancy platform, the peer review system can be accessed by various stakeholders such as engine authors and end-users (healthcare providers with physicians and clinicians, researchers, industry entities, or groups of these). Access control to certain images, image cohorts, end-user physicians or clinician feedback, governance, engines for upload or deletion, engines for running images and clinical content, and user settings are able to be controlled to prevent commingling of images, engines or use without permission from its authorized owner(s). Any stakeholder can be an engine author which can create an algorithm which can be used by an end user, without the end user having access to the algorithm, code, or image data. This can be done by sending the studies to a private or multi-tenant secure server, which can be cloud based or locally sited to process the study with any number of containerized engines/algorithms. The access control allows algorithm developers to grant authentication and administration privileges.

On embodiment of algorithm and use authentication gives algorithm developers the ability to grant different end users the ability to use an algorithm, or allowing them to be published for use publicly while requiring the end user(s) to agree to platform and licensing agreements provided in written form or via click-through legal terms and conditions. While administrative privileges gives an algorithm developer the ability to have other algorithm developers modify the algorithm or create a new version of the algorithm, or engines or engines of engines to modify the algorithm. Version control allows algorithm developers the ability to create different generations of their algorithm, while tracking changes to the algorithms technical file for regulatory clearance. Similarly, different generations of image and clinical content cohorts and peer review feedback data are also versioned and secured to protect the intrinsic value and avoid unintended proliferation of data.

In one embodiment, image processing engines (also referred to as image processing modules or image processing units, which may also process or only process data relating to or unrelated to any images) can be developed by a variety of developers which may be operated by a variety of organizations or enterprise entities. An image processing engine refers to an executable image or binary code that can be individually and independently launched and executed by a processor, in some cases, in combination of hardware processing resources (e.g., graphic acceleration devices such as graphic processing units or GPUs), to perform a specific image process on an image (such as shape recognition, size measurement, etc.). The image processing engines can be uploaded and listed in a Web server to allow a user to select and program the intended operation parameters and/or download one or more image processing engines to run in a specific location independently as an insular locally-sited Peer Review System solution, and/or in communication and combination with another system (in hybrid mode). The selected image processing engines can be configured to a variety of configurations (e.g., in series, in parallel, or both) to perform a sequence of one or more image processing operations.

According to another aspect of the invention, image processing engines can be utilized to inject studies into other commercial or independently-developed peer review systems which are designed to review the medical findings identified by a set of physicians. In one embodiment, the image processing engines are used to confirm or verify findings by the physicians, where the engines operate as peer review systems. The image processing engines can also be utilized to screen and identify any images that are more likely to have abnormal findings and send those for peer review on a third party system, or evoke diagnostic review on the Peer Review System invention.

The identified images are then reviewed by a set of physicians to verify and confirm the findings. In the latter case, the engines operate as preliminary reviewers. As a result, for thousands of medical images that need to be reviewed, the image processing engines can perform massive image processing operations to preliminary identify the abnormal images, and the engines can prospectively “learn” from the feedback when these images are reviewed by the physicians to during diagnostic interpretation. If the findings of the image processing engines and the reviewing physicians are consistent, the operations of the image processing engines involved can be validated, i.e., the algorithms used by the image processing engines are validated. Otherwise, such algorithms may need further fine tune or training, for example, using machine learning methods. Sometimes the function of an engine is called an algorithm. When a physician or engine performs an action or applies an algorithm/input or tool it is sometimes referred to as an operation. These operations result in findings, which are a part of the overall interpretation outcome of a peer review study. Similar to peer review workflow but involving engines, in the Peer Review System, there is a first result (from a physician, engine, engine of engines, or operation), this is compared to a second result (from a physician, engine, engine of engines, or operation) and in the case of disagreement, these are adjudicated by a third result (from a physician, engine, engine of engines, or operation). As such, in the Peer Review System, the enabling technology expands the roles and applications of peer review in novel ways by performing operations either for the interpreting physician, before the physician, or after the physician, and using this interaction to support the comparison of human and machine (engine) found findings and providing a technology platform and method for human input, engines, content and findings to be captured, collated and combined in a real-time image interpretation environment, in addition to typical peer review environments. In the case of the Peer Review System, this includes interaction (synchronously or asynchronously) between any combination of physicians, engines (or engines of engines), content/image cohorts and 3rd party validated data sources.

The physician confirmations and rejections as well as other collectable workflow inputs they provide using the Peer Review System can be used as training data in a closed-loop fashion whereby the engine becomes continuously or iteratively trained using machine learning techniques in a supervised or unsupervised fashion, If there is any inconsistency between the engine findings and the physician findings (including physician adjustments, confirmation, or rejections), a message is sent to the database recording the event, and optionally, a message can be sent to any predetermined device(s), individual(s) or groups even during the primary interpretation process or Peer Review System process indicating that something needs to be paid attention. This may occur in an unsupervised fashion with engines providing feedback to other engines, still creating a closed loop learning scenario. In such cases, the first, second and even third result may be provided from engines (or engines of engines) and not humans, and used for the purpose of enhancing the engine(s) used by the Peer Review System. When the first, second and third result are derived entirely from humans, this is typical peer review and is not a part of the Peer Review System invention. However, in such case, the image and content cohorts of these interpretations with the validated findings can be captured as an image/content cohort and this process is a function of the invention. Such cohorts can be used by engines retrospectively to learn, and these data can be injected into the peer review process by the Peer Review System to further verify the performance of physicians, further improve the image/content cohort, and to develop new engines and/or engines of engines.

Engines (and engines of engines) must perform well on live streams of incoming clinical data and not just the image/content cohorts. These real-time clinical images and content sets that need interpretation are often imperfect. This can occur because there are patient scanning defects such as scanning protocol errors, patient movement, metal artifacts, obese patients, etc. It may also happen due to a lack of availability of prior imaging studies that are related to the current one, or missing clinical information or medical errors, etc. For these reasons, real-time study evaluation is more challenging than processing image/content cohorts and various engines/operations can be expected to fail. The Peer Review System can utilize cohorts of challenged or failed studies to determine the likelihood of any given ensemble, engine or operation succeeding given the use case and quality factors of the data presented. It can utilize this information in an engine of engines that analyzes data and influences which engines, ensembles and operations are run, to best deliver the required/desired findings for any particular study or even a challenging cohort of images. By optimizing this way, the Peer Review System reduces wasted compute power, reduces wasted physician time reviewing inferior findings, and increases the consistency and performance of engines and ensembles, thereby utilizing the intelligence of the Peer Review System to improve the performance of the Peer Review System itself.

Alerts can be provided if there are consistent, inconsistent, normal, abnormal, high quality, low quality, failed, or successful results returned by one or many engines or ensembles. Additionally, a supervised or unsupervised machine learned engine can be used to monitor and learn the effectiveness of various engines in various situations and begin to optimize the use of various engines to be best applied to various use cases in order to increase the number of findings which need to be confirmed by a physician or that are likely to otherwise be missed by a physician if not marked or otherwise measured, mentioned, indicated or marked by the engine and supplied by the cloud platform, whether inside the Peer Review process or inside another physician or clinician image interpretation or review process, or within an electronic health record, viewing system, PACS or 3D advanced visualization system.

According to one embodiment, when a first set of medical images associated with a particular clinical study is received from a medical data source, one or more image processing engines are invoked to process (e.g., recognizing shapes, features, trends in the images or other data or measurements) the medical images (or data, used synonymously in this application) according to a predetermined or machine learned suggested order for performing the engine operations that is configured for the particular type of imaging study. The image processing engines are configured to perform image processing operations to detect any abnormal findings of the medical images, or to optimize clinical workflow in accordance with the preferences or computer-observed working ways of the end-user (based on the system that they are using for interpretation, or in an end-user customized manner as part of the Peer Review System functionality) and to generate a first result describing the abnormal findings or preferred presentation of the images and normal and/or abnormal findings. The physician input represents the second result. The Peer Review System detects agreement or disagreement in the results and findings and sends alerts for further adjudication given the discordant results, or it records the differences and provides these to the owner of the algorithm/engine allowing them to govern whether this feedback is accepted (i.e. whether or not the physician input should be accepted as truth, and whether this study should be included in a new or updated cohort.)

One embodiment of the invention regulates inferencing and image cohort collection based on image acquisition quality. Image quality needs to be checked and verified before or after any predictive engine is evoked in order to ensure engine standards are met. This may include the standards of regulatory and oversight bodies responsible for quality control in imaging informatics. One example of this pertains to pulmonary embolism studies. Sensitivity and specificity for detection of a pulmonary embolism is directly related to image acquisition quality. Artifacts that result in image degradation such as respiratory motion artifact or technical acquisition parameters (e.g. contrast bolus timing) directly impact the ability of an engine to confidently identify a given finding. For a pulmonary embolism detection engine result to be presented to the physician or verified, a quality control engine must assess contrast bolus timing and for respiratory motion artifact in order to modify the confidence of the pulmonary embolism detection engine. There may be engines that perform better or worse given the presence or absence of a given artifact which can be automatically selected to process the images. The combination of engines processed ensures the optimal and appropriate confidence of the finding output for a given finding. The presence or absence of a finding may therefore result as a range or function of the study quality and not necessarily a discrete value. This is one embodiment of the engine of engine selector with respect to quality control and handling of image artifacts and technical image acquisition quality variations.

A similar quality control paradigm applies to image cohort curation quality scoring. For each image stored in a given image cohort that is curated by a combination of physicians and engines, quality scores with the presence and absence of imaging artifacts are stored in a database along with findings. The automated quality control score can be accepted or rejected by a diagnostic image interpreter or provider. Both high and low quality label sets are curated. A given engine's performance is scored against both high and low quality data sets to determine if an engine can be used if certain artifacts are present.

Specifically in one embodiment of the invention, the image processing engines are provided by one or more image processing engine developers which may be operated by the same or different entities or organizations. A second set of medical images is transmitted to a first review system, wherein the second set of medical images is a subset of the first set of the medical images. In one embodiment, the second set of medical images have been categorized by the image processing engines as abnormal images. The review system operating as a peer review system is configured to review the second set of medical images to verify or confirm or unverify or reject the abnormality of the images, generating a second result. In response to the second result received from the review system, the operations of the image processing engines run on the Peer Review System are validated or invalidated based on the first result and the second result (this is done on the 3rd party conventional peer review system or on the Peer Review System invention described herein, which has such capability.

Machine learned engines may learn from this information and/or the governance and/or owner of the algorithm may accept or reject such feedback into the learning process, or an unsupervised engine may experiment with various scenarios on its own to find a statistically ideal combination of accepting some feedback and rejecting other feedback. Engine(s)/ensemble(s) performance can be verified against a reference standard image cohort that is not available to an engine author as training data in order to perform quality control, wherein versioning of an engine displays these performance metrics and/or versions a given algorithm and the cohorts of images provided to an author for traceability in accordance with typical healthcare regulatory and reference standards. The injected images or image cohorts specially assembled for algorithm verification and engine qualification are randomized in terms of the number and types of images provided to prevent overfitting of a given version of the algorithm (i.e. adjusting the data to make the algorithm look good or rate well). Such versioning ensures a defined separation between training data and validation data, in the case where it is not appropriate to perform validation using a reference standard cohort.

In one embodiment, by validating an image processing engine's results consistently and by many users, the image processing engine may become a “certified” or “approved” image processing engine by utilizing these data to support regulatory certifications, by an outside third party entity such as the FDA, or similar. If an image processing engine cannot be validated based on its results of use, the parameters or algorithm of the image processing engine may need to be adjusted or retrained, for example, using a machine-learning method based on these prior results (image cohorts and clinical content cohorts). Further, the revisioning of engines, image cohorts, clinical cohorts and the saving of all Peer Review System utilization data provides a method for engines of engines to learn and adapt, and for engine authors to improve the performance of their engines based upon these recorded events.

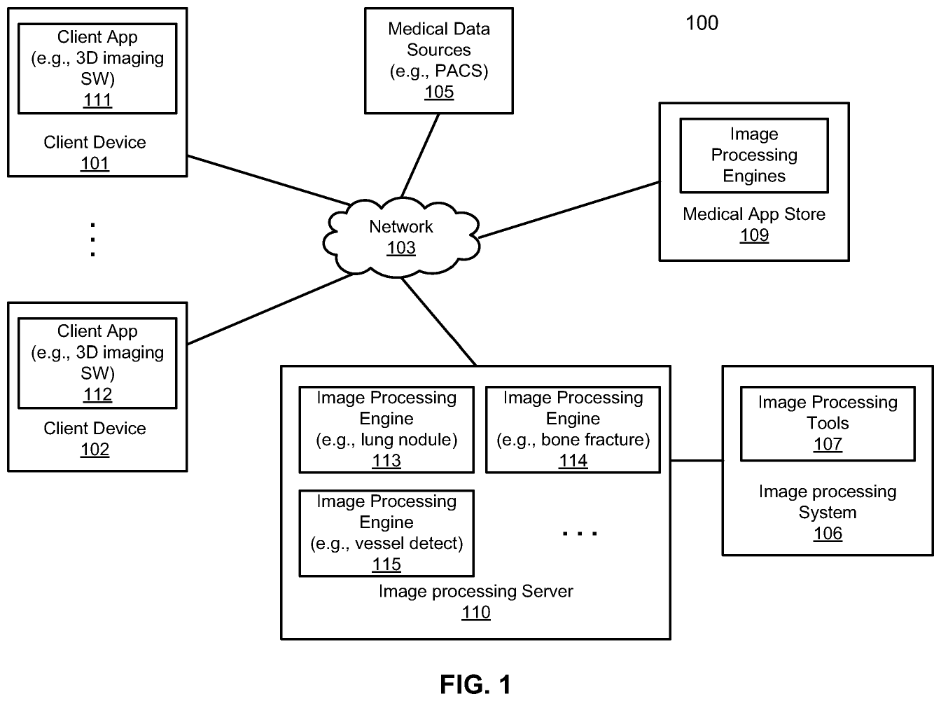

FIG. 1 is a block diagram illustrating a medical data peer review system according to one embodiment of the invention. Referring to FIG. 1, medical data peer review system 100 includes one or more client devices 101-102 communicatively coupled to medical image processing server 110 over network 103. Client devices 101-102 can be a desktop, laptop, mobile device, workstation, etc. Network 103 may be a local area network (LAN), a metropolitan area network (MAN), a wide area network (WAN) such as the Internet or an intranet, a private cloud network, a public cloud network, or a combination thereof.

Image processing server 110 hosts a number of image processing engines 113-115 that can be invoked and configured to perform a series of one or more image processing operations or clinical content processing operations on medical images and clinical content, which may be provided by medical data sources 105. Medical data sources 105 can include Laboratory Information System (LIS), Radiology Information System (RIS), Enterprise Content Management Systems (ECM), Electronic Medical Record (EMR), Hospital Information System (HIS), Picture Archiving and Communication System (PACS), VNA (Vendor Neutral Archive), Advanced Visualization 3D systems, EMR data, various directories as well as other data sources such as HIE (health information exchange) servers and individual or organizational repositories. Medical data sources 105 may be managed and/or operated by different organizations or information providers than the organization which operates the image processing server 110. Medical image data source 105 can include image data from cloud based storage, local drive, CD, hard drive, DVD, USB, web uploader, any DICOM repository or source, other sources of images and clinical content, or any combination thereof. The image processing server 110 can receive image data (e.g., studies, clinical reports, images, patient data, utilization data, or any combination thereof) over a network from the medical image data sources 105.

The Peer Review System recognizes the intrinsic value of labelled data, which requires human intelligence and verification, or the intentional collection of large amounts of unlabeled data. As an option to prevent reverse engineering of engines by way of labelled data theft, or to prevent duplicating a labelled data set by stealing an engine that can perform this task, the peer review system includes a watermarking, image labelling and/or encryption capability with or without an underlying certificate or verification system, which can be utilized to prevent access, running, decrypting or export of labeled data, source data, or restrict engines/ensembles from running in the absence or presence of such marking without engine author permission.

One embodiment of the Peer Review System can protect the authors of an engine's intellectual property by preventing the reverse engineering of an engine by collecting annotated data without permission of an author. This may vary based on EULA and permissions set by the author and end user. Several sample implementations of this feature include but is not limited to a) tracking of studies by annotating metadata or image data using a block chain based (e.g. etherum) DApp (decentralized application) b) watermarking image overlays generated by engines c) encrypting the output of an engine to be viewed with an authenticated viewer or PACS environment d) preventing bulk data export of annotated image data and or metadata e) logging use of annotated image cohorts, f) preventing the running of an engine/ensemble without the receipt of a verification certificate, and g) preventing an engine from running on data unless it contains specific markings or annotated metadata, or an encrypted access key, etc.

In one embodiment, the medical data provided by data sources 105 may include medical image data in a DICOM format, medical image data in a non-DICOM format, scheduling data, registration data, demographic data, prescription data, billing data, insurance data, dictation data, report data, workflow data, EKG data, best practices reference materials, reference materials, training materials, etc. These data may reside in several locations or systems including HIS, RIS, PACS, LIS, ECM, EMR or other systems. The non-DICOM data may be in several formats including A/V, MPEG, WAV, JPG, PDF, Microsoft Office™ formats and other formats. Generally, data in a PACS will include DICOM data, where data in the HIS, RIS and LIS, ECM, EMR will include non-DICOM data, including both image and non-image data. HIE data includes data available via a health information exchange system. These data generally include data available across different organizations within a region, community or hospital system, and may be text-based, file-based, DICOM or non-DICOM image data. Other data may include any other relevant data, including data in directories on computers, databases, white papers and clinical repositories, research institutions and data collected from users, mobile devices, and in the course of clinical use, etc.

Image processing engines 113-115 can be developed and provided by a variety of vendors, which may be operated by a variety of organization or enterprise entities. One embodiment is an image processing engine as an executable image, container, virtual environment or binary code that can be individually and independently launched and executed by a processor, in some cases, in combination of hardware processing resources (e.g., graphic acceleration devices such as graphic processing units or GPUs), to perform a specific image process on an image (or data set, used synonymously), such as trends, comparisons, specific values, characteristics, shape or likeness (similarity) recognition, areas of interest, size, measurements, etc. The image processing engines 113-115 can be uploaded and listed in a Web server 109, in this example, an application store, to allow a user of clients 101-102 to purchase, select, and download one or more image processing engines as part of client applications 111-112 respectively. The selected image processing engines can be configured to a variety of configurations (e.g., in series, in parallel, or both) to perform a sequence of one or more image processing operations. The image processing engines 113-115 can be downloaded to client systems 101-102 to perform the operations. Alternatively, the image processing engines 113-115 can be hosted in a cloud-based system, such as an image processing server 110 as a part of software as a service (SaaS) and/or platform as a service (PaaS), to perform the operations and allow authors of engines to control access and maintain versions and regulatory compliance.

In one embodiment, each of image processing engines or modules 113-115 may be configured to perform a specific image processing operation on medical images, such as, for example, lung nodule detection, bone fracture detection, organ identification and segmentation, blood clot detection, image body part categorization, chronic obstructive pulmonary disease (COPD) detection, or soft tissue characterization. An image processing engine can perform such a detection based on the shape, texture, sphericity measurement, color, or other features obtained from the medical images or which are derived or implied by the clinical content. In one embodiment, multiple image processing engines provided by multiple vendors can be configured in series, in parallel, or a combination of both to perform image processing operations, which may be configured via a configuration interface of medical image processing server 110 or through client applications 111-112.

In one embodiment, when any one of image processing engines 113-115 is invoked, it may further invoke one or more image processing tools 107 of image processing system 106, which may be integrated as a part of image processing server 110 or alternatively, as a remote medical image processing system (or a cluster of systems or servers) communicatively coupled to image processing server 110. Image processing system 106 may be implemented as part of a TeraRecon® AquariusNET™ server and/or a TeraRecon® AquariusAPS™ server. Each image processing engine may invoke medical image processing system 106 to perform an image processing operation on an image of a body part of the patient which was navigated to or may be automatically detected by an engine or engine of engines, to produce certain image quantitative data or measurement data on such images. Similarly, clinical content may be investigated.

The image quantitative data may be used to manually or semi-automatically determine or measure the size and/or characteristics of a particular body part of the medical image. The image quantitative data may be compared with a corresponding benchmark associated with the type of the image to determine whether a particular medical condition, medical issue, or disease is present or suspected. The likelihood of such occurrence may further be predicted or determined based on a trend of same type of medical data of the patient as part of medical history of the patient and/or other patients' data. In one embodiment, Ensemble engines may be combined, for example, one which finds the body part, another that segments it, another that labels the anatomy within it, and another that detects signs of leading diseases in that area, and finally another that can match these findings with clinical information resources and recommendations to provide assistance and direction to the physician.

In one embodiment, a processing engine may be associated with a particular body part of a patient. Only certain engines will apply according to what part of the body it is pertaining to, or according to what imaging modality type (the imaging procedure type) that is used. This will help the engine of engines mentioned above make a good choice and learn what works.

The image processing server 110 can further include one or more e-suites (i.e., e-suites, also referred to as ensembles, can be a combination of one or more image processing engines). As such, ensembles can be cascaded for increasing granularity, thereby increasing sensitivity and specificity for the particular intended action of the ensemble engine.

The engine or e-suites can detect findings (e.g., a disease, an indication, a feature, an object, a shape, a texture, a measurement, insurance fraud, or any combination thereof). The one or more engines and/or one or more e-suites can detect findings from studies (e.g., clinical reports, images, patient data, image data, metadata, or any combination thereof) based on metadata, known methods of in-image analysis, or any combination thereof. The image processing engines 113-115 of image processing server 110 can flag image data with findings, for example, indicating that the image data is abnormal.

Flagging can include displaying the actual finding(s), or derived summary indication utilizing a combination of findings, found by the engine/e-suite, the engine/e-suite name, a simple mark representing that the study was processed by the image processing server 110, marking that the study was normal/abnormal, a series of macro level indication choices (e.g., red, yellow, green, or orange) depicting the risk of the findings, marking with severity (e.g., mild, moderate, or severe) or icons denoting a finding (e.g., a simple mark denoting that there is a findings), relevant tools automatically evoked in the image viewing system, or any combination thereof, or otherwise, as provided by the engine/e-suite/ensemble or engine of engines.

Flagging can occur on the study or separately from the study. Flagging may be available and accessible through one or several restful services, API's, notification systems or pushed to a third party application or on image processing server, or the database(s) of the Peer Review System. In one embodiment, flagging can be displayed or viewed in a 3D medical imaging software application (e.g., client applications 111-112). The engines and/or the e-suites can machine learn or be trained using machine tearing algorithms based on prior findings periodically such that as the engines/e-suites process more studies, the engines/e-suites can detect findings more accurately. In other words, the confidence level of detecting findings increases as more studies are processed. Based on the findings of the engines and/or e-suites, the image processing server 110 can prioritize and sort study worklist based on, for example, type of findings, severity of findings, risk to patient health, or any combination thereof. This is the final output of the platform including a list of results and macro findings that can be used in the process of primary image interpretation, and any of these findings can be opened or further interrogated in terms of the underlying assumptions for adjustment or the quality of the image data or clinical data can be assessed for appropriateness and possible exchange or editing.

An interface with restful services, or an API, provides bidirectional communication between the Peer Review System, other common peer review systems, and other medical image viewers, such that any feedback provided in these 3rd part applications can be returned to the Peer Review System to facilitate engine learning and the curation of additional image data cohorts and clinical content cohorts.

The application store 109 may be an e-commerce server that can store one or more engines, one or more e-suites, or any combination thereof. The image processing server 110 can store the same or different engines or e-suites as the application store 109. The engines or e-suites of the image processing server 110 can process studies depending on which engines are selected by the user via a graphical user interface (GUI) or website (local or on the Internet) of the image processing server 110. The image processing server 110 can send updated/improved engines or e-suites to the application store 109. The application store 109 or the image processing server 110 can store user profiles and/or group profiles. The user profiles can be specific to one or more users. The group profiles can be specific for one or more groups, for example, a governance board, a radiologist group, a cardiologist group, a technician group, a developer group, or any combination thereof. The user profiles and the group profiles can have access controls to tools, engines, e-suites, training tools, coding tools, or any combination thereof. Users and/or groups can expand or reduce access control to other users and/or groups.

Tools, engines, e-suites, training tools, coding tools, or any combination thereof can be displayed and used via the image processing server 110 or in a 2D and/or 3D medical imaging software application, or peer review system, or the novel Peer Review System. A medical imaging software application is a client application that accesses the output of the image processing tools 107 of image processing system 106. For example, a first user can upload a first engine via the client device (e.g., a website, mobile phone, a workstation, a computer, an iPad, a laptop, or any other method or type, or combination thereof) that can be stored in the application store 109. The first user or a governance board can provide access to certain tools, for example machine learning/training tools, to a second user or group. The second user or group can use the machine learning/training tools and the feedback from this usage can be applied to train the first engine to detect findings with higher accuracy. The first engine can be updated by the image processing server 110 and stored in the application store 109. The processing of image data by engines and updating of the engines can occur at the image processing server 110, the image processing application store 109, or any combination thereof.

Note that these engines can have measured performance attributes that are either prescriptive, implemented through supervised learning, or allowed to be self-developed by the engines (engines of engines) through either supervised or unsupervised learning. Then, through governance, the person uploading the engine can either accept or reject the changes and/or publish them for use by others, or not.

The image processing server 110 can comprise of engines or e-suites from one or more medical institutes at different locations, one or more users, one or more groups, or any combination thereof. The image processing server 110 can have a graphical user interface (GUI) such that one or more users can upload or download one or more engines or one or more e-suites.

The image processing server 110 can have a GUI for one or more users or groups to train, code, develop, upload, delete, track, purchase, update, or process data on engines or e-suites. The image processing server 110 can have access controls. The image processing server 110 can be password protected to support a multi-tenant environment which provides independent security and cloud access controls to support controlled access to engines, ensemble engines, engines of engines and the configuration thereof. These passwords (and or authentication methods for integrated workflow with other systems) support separate access control of image cohorts, clinical data cohorts, engine accessibility and the interaction database.

The image processing server 110 can allow users or groups to give access control to tools and engines to other users or groups from the same medical institute or other medical institutes (e.g., multi-tenancy configuration). This can promote collaborative efforts among one or more users or groups from one or more medical institutes to improve engines or e-suites to detect findings. The image processing server 110 can have a GUI that can allow the users to run engines ore-suites to process image data. In one embodiment, the output of the Peer Review System can be integrated and/or consumed by any third-party system that supports the restful and or API communications or is capable of read/write to the database of the platform. Alternatively, the viewing portion of the Peer Review System may be the consumer of this content and/or be embedded into the third party system or used as stand-alone. Any image processing engines 113-115 can further invoke image processing tools 107 of image processing system 106, depending upon the settings for security and governance which are applied by the engine owners and the Peer Review System users performing peer review and/or diagnostic interpretation.

An engine author can upload any image processing engine or e-suite through the image processing server 110 to the application store 109 using its graphical interface such as web interface. The image processing server 110 can have a developer platform for one or more engine developers to update, change, train, machine-learn, or any combination thereof any of the engines on the image processing server 110. The engines can be improved to detect the findings on a developer platform, for example, via training using machine-learning algorithms or via modifying a containerized version of a predict method for a given engine. One way this approach can be accomplished is by aggregating data to improve a given engine and versioning the data used for iterative training assessment and the versioning of engine source code within a given software container or wrapper asynchronously, allowing distribution and updating of algorithms in use either in the cloud or which are in use remotely in a deployed containerized algorithm player software that is administered and governed by the actions of the end-users and algorithm/engine authors collaborating and working in the platform.

One or more individual processing engines can be wrapped in a software container with a defined set of inputs and outputs. Processing engines having compatible inputs and outputs can be executed serially or in parallel (e.g., via multiple threads) to create a more accurate final output. In one embodiment, a standardized restful web services API (or similar) may be utilized that allows the abstraction of the needed inputs and outputs of a specific engine/algorithm to the standard published schema as supported and updated by the platform. This requires every engine to have an abstraction layer on the input and output side that allows the mapping and abstraction. Then one or more abstracted outputs can be mapped to one or abstracted inputs.

For example, an engine developer can train a lung nodule detection engine to detect lung nodules in studies on the developer platform of the application store 109 or a data analytic system (not shown) by training the engine based on various features of detecting lung nodules (e.g., geometric shapes, textures, other combination of features resulting in detection of lung nodules, or any combination thereof). In another example, the engine developer can train a blood clot engine on the developer platform. In another example, a bone fracture engine can machine-learn on the developer platform based on bone fracture engine data from the image processing server 110. In another example, a COPD engine can machine learn based on the same COPD engine data, based on another COPD engine data, or any combination thereof.

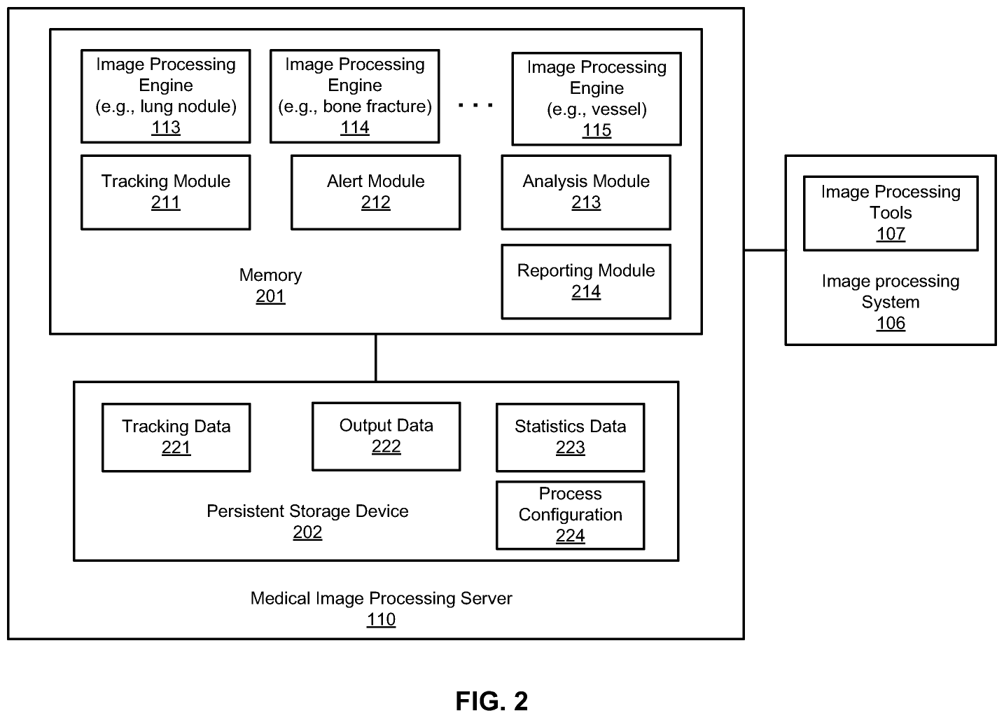

FIG. 2 is a block diagram illustrating an example of an image processing server according to one embodiment. Referring to FIG. 2, image processing server 110 includes memory 201 (e.g., dynamic random access memory or DRAM) hosting one or more image processing engines 113-115, which may be installed in and loaded from persistent storage device 202 (e.g., hard disks), and executed by one or more processors (not shown). Image processing server 110 further includes tracking module 211, alert module 212, analysis module 213, and reporting module 214. Image processing engines 113-115 can be configured in a variety of configurations according to process configuration 224. Process configuration 224 may be stored in a configuration file that is specifically configured by a user or for a particular study or images. Medical image processing server 110 may be multi-tenancy cloud server. There may be multiple configuration files, each may be associated with a user or a group of users, which may be configured via a configuration interface (not shown).

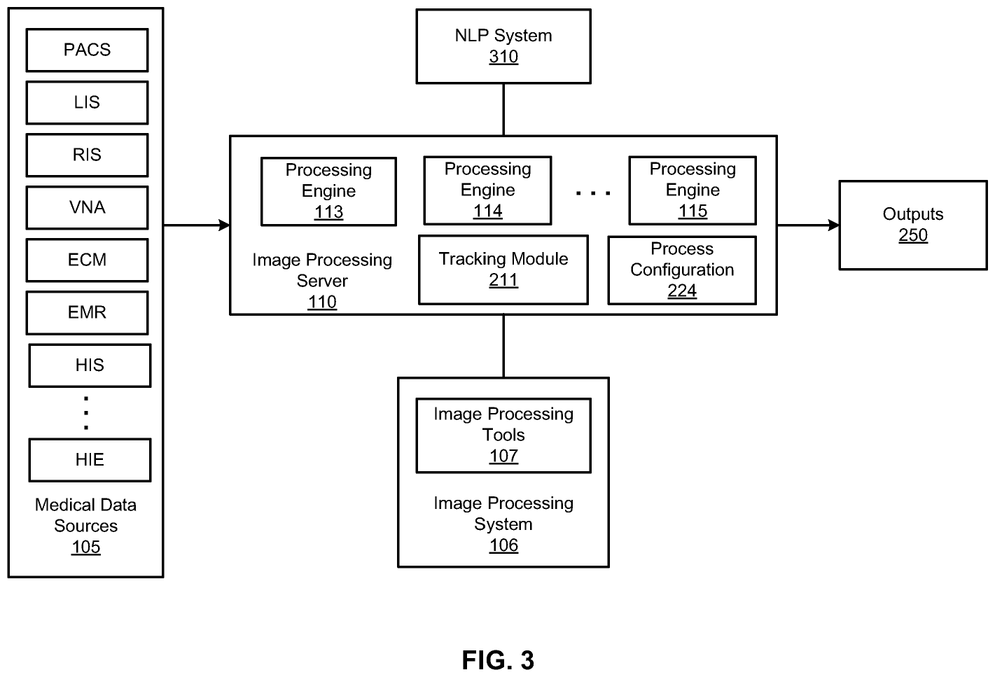

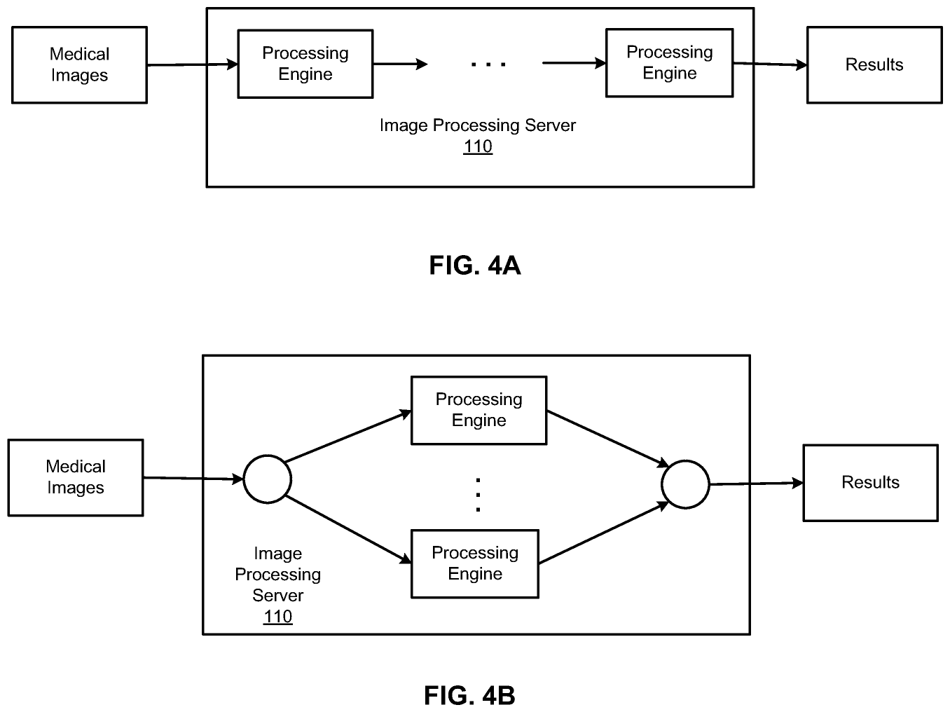

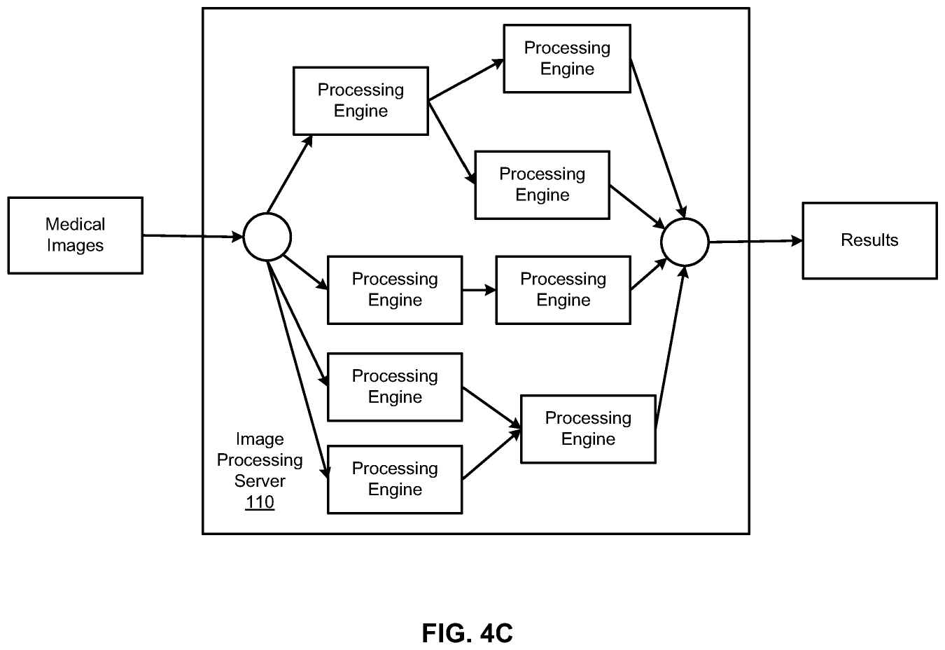

For example, referring to FIGS. 2 and 3, medical data (in this example, medical images) can be received from medical data source 105 at image processing server 110. One or more of image processing engines 113-115 can be arranged according to a sequential order based on process configuration data 224. The image processing engines 113-115 may further invoke image processing tools 107 of medical image processing system 106. One or more results 250 may be generated and stored in persistent storage device 224 as a part of output data 222. In one embodiment, image processing engines 113-115 can be arranged in series, in which an output of a first image processing engine can be utilized as an input of a second image processing engine, as shown in FIG. 4A. Alternatively, image processing engines 113-115 can be arranged in parallel to perform the same or different image processing operations concurrently as shown in FIG. 4B. The outputs of the image processing engines are then aggregated to generate a final result. Furthermore, image processing engines 113-115 can be arranged in both series and parallel as shown in FIG. 4C.

In one embodiment, image processing server 110 may further invoke a natural language processing (NLP) system 310 to process the texts or languages of outputs 250. NLP system 310 is able to scan, analyze, and match features extracted by image processing server 110 to identify studies with missed findings or misinterpreted findings to correlates with outputs 250. NLP is a field of computer science, artificial intelligence and linguistics concerned with the interactions between computers and human (natural) languages, and, in particular, concerned with programming computers to fruitfully process large natural language corpora. Many different classes of machine learning algorithms have been applied to NLP tasks. These algorithms take as input a large set of “features” that are generated from the input data.

For example, a first engine can run an algorithm to detect findings. The first engine can create an output of the findings of the first engine. The findings by the first engine can be included in the statistical interface, a report (not shown), in a diagnostic interpretation viewer (not shown, or any system or database capable of accessing results and cohorts through the restful services and/or APL A physician can review the output of the findings. The physician can validate/invalidate the findings of the first engine. The validation/invalidation of the findings of the first engine can be included as part of output data 222. The first engine can process studies from one or more medical institutes where the results can be included output data 222.

Any stakeholder can be an engine author which can create an algorithm which can be used by an end user, without the end user having access to the algorithm, code, or image data required to train the algorithm. This is done by sending the studies to a private or multi-tenant secure server, which can be cloud based or locally sited to process the study with any number of containerized engines/algorithms. The access control allows algorithm developers to grant authentication and administration privileges. On embodiment of algorithm and use authentication gives algorithm developers the ability to grant different end users the ability to use an algorithm, or allowing them to be published for use publicly while requiring the end user(s) to agree to platform and licensing agreements provided in written form or via click-through legal terms and conditions. While administrative privileges gives an algorithm developer the ability to have other algorithm developers modify the algorithm or create a new version of the algorithm, or engines or engines of engines to modify the algorithm. Version control allows algorithm developers the ability to create different generations of their algorithm, while tracking changes to the algorithms technical file for regulatory clearance. Similarly, different generations of image and clinical content cohorts and peer review feedback data are also versioned and secured to protect the intrinsic value and avoid unintended proliferation of data.

In one embodiment of this invention a post contrast CT scan of the abdomen is processed prior to a CT fluoroscopy procedure. The post contrast images are registered to a CT fluoroscopy data set using a registration engine. The results of the registration and anatomic segmentation can be toggled over the CT fluoroscopic data in order to display blood vessels on a non-contrast CT fluoroscopic image during a CT guided biopsy or ablation. Thus resulting in virtual contrast enhanced fluoroscopic results. This may be supported similarly with other modalities, such as MRI. In one embodiment, the validation or invalidation of the output of findings of thee-suite can be included in tracking data 221 and/or statistics 223.

According to another scenario, for example, a PACS server or CT, MRI, ultrasound, X-ray, or other imaging modality or information system can send studies to a first engine of the e-suite. After the first engine processes the studies, the output of findings from the first engine can be sent to a second engine and a third engine. The second engine and the third engine can run in parallel. The output of findings of the second engine and the third engine can be combined. The combined output of the second engine and the third engine can become the output of findings of thee-suite. Alternatively, the process may begin with multiple engines receiving the data for processing and these send their results to one or more other engines as described. The final output can be sent back to the source modality, or a PACS, or the Peer Review System to be reviewed by a physician to confirm or deny the findings of the output of the e-suite ensemble.

The output of the first engine can have a first weight factor. The output of the second engine can have a second weight factor, etc. The first weight factor and the second weight factor can be any percent ranging from −100%% to +100%, or a logarithmic scale, or any author-assigned scale of any kind that is appropriate for the type of test and cohorts being run. The weighted output of findings can enable one engine to have more weight than another engine, and one type of finding in an engine can have different weightings for each finding. The user can manipulate the weights of each engine from an interface on the image processing server. Alternatively, the engine of engines can be applied to set these values using supervised or unsupervised machine learning techniques.

For example, the first engine can be an engine for edge detection. The second engine can be an engine for soft tissue detection. A user can manipulate each engine such that the first engine is weighted at 20% and the second engine is weighted at 80%. The output of the first and second engines can reflect such weights. Multiple engines or e-suites can be run at the same time, in parallel, for identical studies. Multiple engines or e-suites can be run at the same time for different studies from the same patient or from different patients.

Similar engines which find similar findings can be run in parallel, in series, or any combination thereof can be different engines to detect the same finding. For example, a first engine, a second engine, and a third engine can be lung nodule detection engines, but they can be from different engine developers or different medical institutions. Such a configuration can enable comparing the findings from the three engines from different vendors, providing physicians with immediate access to multiple tools and a quick overview of the findings from each engine, immediately during the diagnostic interpretation process which occurs during peer review. Alternately, the Peer Review System can allow the diagnostic review to occur in the common PACS system and then the peer review to occur after such review using the Peer Review System to measure similar and different findings between these diagnostic interpretations.

The difference between typical peer review and the Peer Review system is that common peer review only seeks to confirm agreement about the overall result of the interpretation, whereas the Peer Review System allows for measurement of agreement on a more granular level, including findings, and therefore provides the detail necessary to train an image processing or data processing engine to provide improved future results. While the Peer Review System may require more physician engagement time when it is initiated, the availability of highly tuned algorithms will be a result of continued use, and that will reduce the overall physician interpretation time in the future, and provide for improved peer review results over time.

According to one embodiment, a processing engine can analyze multiple studies with a similar modality and produce an analysis result of “no significant interval change.” For example, a processing engine can take two head CT studies, which occur at different times, but of the same modality and body part. An easily extracted report feature from the follow study is “no significant interval change.” A processing engine would then run on both CT studies to compare the two to see if there are any differences. If the most recent report is deemed “no significant interval change,” a Peer Review System function can be to run an engine that can verify the similarity, and therefore provide the ability to agree or disagree with that statement. Often, the reported findings are maintained in reports, and electronic communications, which are inputs to the platform and the relevant contents are provided to the engine when it runs.

According to another embodiment, an engine running on a single study may call another engine or engines to run on a relevant comparison in order to assess stability of the abnormality. This may be multimodality, such as comparing a CT liver lesion engine to an MM liver lesion engine on a comparison study. In this embodiment, a processing engine running a CT algorithm running on a CT image cohort calls another processing engine or the prior results of an inferenced engine running an MRI algorithm on an MRI image cohort for the same patient, to perform a single comparison task.

The engines and/or e-suites can be run in any configuration such that the probability to detect the findings (or to confirm no findings if that is the goal) is high and/or optimized. The engines and/or e-suites can be run in any configuration to maximize the confidence level to detect the findings. The engines can be run in any configuration such that a user can configure how the output of findings look like (e.g., high probability to detect the finding, low probability to detect the finding, exclude certain findings, include certain findings, selecting normal studies, or any combination thereof).

For example, if a user wants to detect COPD and/or features of COPD, the user can configure one or more COPD engines in parallel, in series, or any combination thereof (i.e., a COPD e-suite), such that the configuration of the engines can have a high probability of detecting COPD or features of COPD. This is an ideal use case for an engine of engines which can self-optimize the weighting of the detection algorithms if it is provided information in the report about which patients did have the targeted findings as confirmed by the physician. As more physicians use the COPD e-suite and confirm (i.e., validate) the output of findings of the COPD e-Suite, the higher ratings the e-Suite can have. High ratings and/or increased confirmations can allow other physicians to recognize which COPD e-suites has the best findings detection rates. This will be evident to users by providing a rating system in the e-commerce site.

In another example, if a user wants to detect lung nodules, the user can select engines that detect specific features of lung nodules, for example, an engine for texture, and engine for nodule shape, an engine for intensity, or any combination thereof. Such engines can be run in parallel, in series, or any combination thereof. Since many lung scans have findings, the most important thing to detect is findings that are likely to result in ordered follow up from the physician. As such, the engine or the engine of engines can be provided the report information or other clinical information in order to improve its detection of lung findings which are most likely to require follow up. Since incidental findings are not missed findings, then one embodiment of the invention is a system that filters out incidental findings in the peer review and diagnostic interpretation process, either by not presenting these findings, or by presenting them and marking them as being likely incidental. Another way to embody the aforementioned process is as a clinical severity score as some incidental findings may or may not have clinical relevance, which affect clinical outcomes. A user can manually replace an engine with another engine through a configuration interface of the image processing server 110 (not shown).

Referring back to FIG. 2, according to one embodiment, tracking module 211 is configured to track or record the assignments and processes of image processing engines 113-115. Note that image processing engines 113-115 can be executed in multiple instances (e.g., multiple threads) in a multi-tenancy operating environment. In the multi-tenancy operating environment, different users can log in and be authenticated. Once the users are authenticated and authorized, the users can configure and utilize the image processing engines according to their service or subscription agreements for different studies, different organizations, etc. Tracking module 211 is configured to keep track of which image processing engines are utilized for which medical studies or by which users, on which image cohorts and clinical content cohorts, which resulted in which indexed user data, then generating tracking data 221 (also referred to as engine data) stored in persistent storage device 202, also called a database or databases.

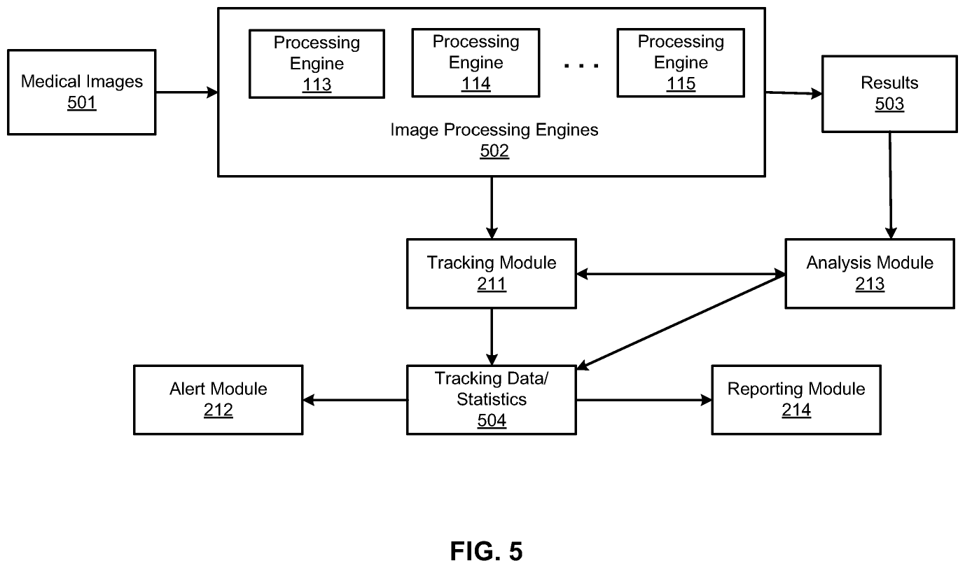

FIG. 5 is a processing flow of processing medical images according to one embodiment. Referring to FIG. 5, when medical images 501 are received, processing engines 502 having one or more of image processing engines 113-115 are configured to process the medical images and to generate results 503. Note that images 501 may represent multiple images within a single study, multiple series within a study, or a combination of series and images from multiple studies of different modalities. Results 503 is analyzed by analysis module 213 to generate statistics data. Meanwhile tracking module 211 is configured to track the operations of processing engines 502. Results 503 may be stored as a part of output data 222. Tracking data and statistics data 504 are generated which may be stored as a part of tracking data 221 and/or statistics 223. Based on the tracking data/statistics data 504, if there is any data satisfying a predetermined condition, such as abnormal findings or inconsistent findings, alert module 212 is configured to generate and send an alert to a predetermined device, database(s) or system(s). A report can also be generated based on tracking/statistics data 504 by reporting module 214.

The tracking module 211 can track the engine data (e.g., which studies have been sent to the image processing server; which studies have been processed by which engines; which studies have been flagged by which engines; which studies have been flagged by multiple engines; which studies have been sent as part of the peer review samples; engine name; findings; data on validated and/or invalidated machine learned possibly higher likelihood of disease studies; data on features of the images in the studies including, but not limited to, wall thickness, texture, slope, measurements, density, heterogeneity, standard deviation of a range of voxels, or any combination thereof; user interactions of the interpreting physicians, as well as any other persons using the system; time for diagnosis; flagging of studies based on, for example, risk to patient health; order of studies based on risk; or any combination thereof) for the one or more engines (e.g., the first engine). Engine data can be tracked and updated manually, continuously, or automatically after each study is run by one or more engines or e-suites. The peer review function may involve more than one, two or three physician interpretations, and the Peer Review System may be used for serial diagnostic interpretation of unrelated studies by a physician or clinical trial.

In addition, analysis module 213, also referred to as a statistical data engine, can perform an analysis on the tracked engine data for an image processing engine. The statistical data engine 213 can aggregate engine data from one or more image processing servers and one or more databases associated with the Peer Review System as well as outside sources, including from one or more medical institutions which provide the engine, and others who only provide image and clinical content cohorts. The statistical data engine 213 can update the statistical data for the all engines and engines of engines based on the engine data, which can be updated on application store 109 as a part of engine ratings. The statistics data may also be stored in persistent storage device 202 as a part of statistics data 223. Similar feedback is collected and displayed for image cohorts and clinical data cohorts.

Note that some or all of the components as shown and described above may be implemented in software, hardware, or a combination thereof. For example, such components can be implemented as software installed and stored in a persistent storage device, which can be loaded and executed in a memory by a processor (not shown) to carry out the processes or operations described throughout this application. Alternatively, such components can be implemented as executable code programmed or embedded into dedicated hardware such as an integrated circuit (e.g., an application specific IC or ASIC), a GPU (Graphics Processing Unit), a digital signal processor (DSP), or a field programmable gate array (FPGA), or similar, which can be accessed via a corresponding driver and/or operating system from an application. Furthermore, such components can be implemented as specific hardware logic in a processor or processor core as part of an instruction set accessible by a software component via one or more specific instructions.

According to another aspect of the invention, image processing engines can be utilized as a part of peer review systems to review the medical findings performed by a set of physicians. The image processing engines are utilized to screen and identify any images that highly likely have abnormal findings. The identified images are then reviewed by a set of physicians to verify and confirm the findings. As a result, for thousands of medical images that need to be reviewed, the image processing engines can perform massive image processing operations to preliminary identify the abnormal images. Those images are then reviewed by the physicians to confirm the findings. If the findings of the image processing engines and the reviewing physicians are consistent, the operations of the image processing engines involved can be validated, i.e., the algorithms used by the image processing engines are validated. Otherwise, such algorithms may need further fine tune or training, for example, using machine learning methods.

Alternatively, if there is any inconsistency between the machine findings and the physician findings, an indication in the database(s) and notifications in the restful services and/or API have the effect of sending notification to the desired systems and staff. These identified conflicting studies are then sent for secondary physicians review. Once both review results are known, reconciliation through the analysis module can result in confirmation of the engine accuracy or improvement to the engine.

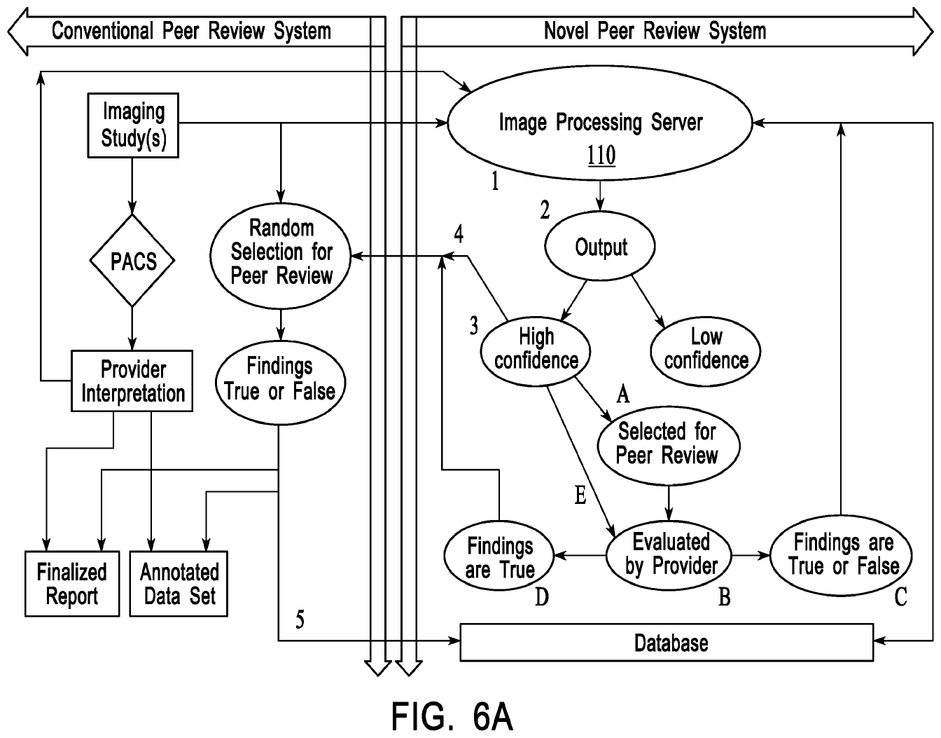

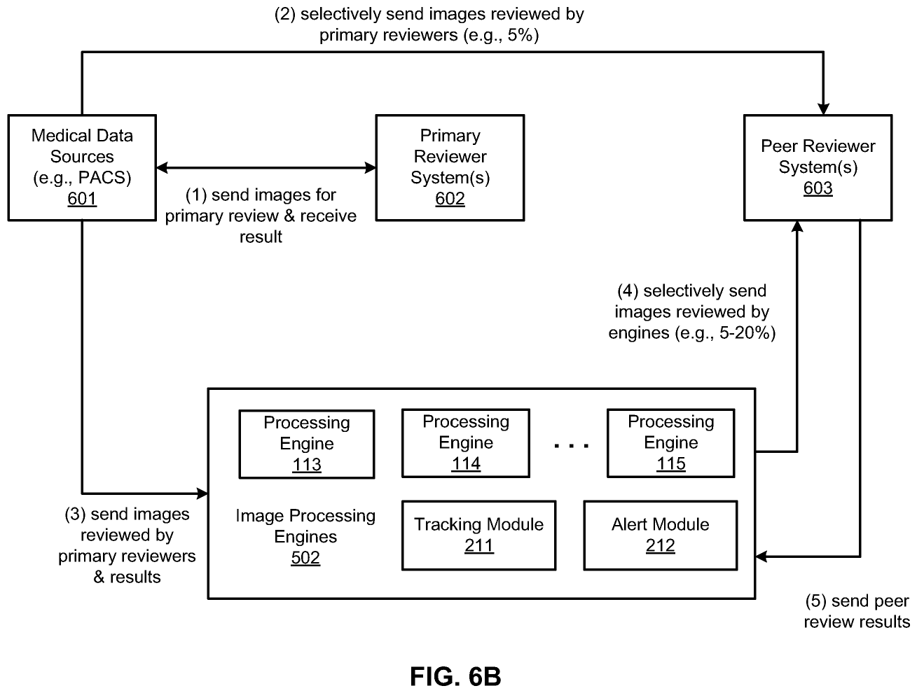

FIG. 6A shows a workflow loop diagram demonstrates examples of novel workflows of the Peer Review System according to one embodiment. Referring to FIG. 6A, the workflow includes a peer review high confidence injection workflow loop. During this workflow loop, the Image Processing Server (110) is initiated by imaging studies or image studies and provider interpretation (also noted as a report) arriving at the Image Processing Server notes as step 1. After a plurality and combination of engines process the image study, the output is noted as step 2. Studies or images with findings determined to have a high confidence of a potential finding or potential discrepancy with the provider interpretation are injected into the selection for peer review in Step 3. In step 4, this injected study is evaluated by a physician (who did not make the initial provider interpretation, if applicable). The results of that interpretation along with the Peer Review System interpretation are stored in the database as step 5 and can be used for future training of engines and engines of engines within the Image Processing Server and Peer Review System. In addition, in Step B, user interaction data is stored in the database as well.

The workflow further includes a peer review injected physician confirmed findings workflow loop. During this workflow loop, the Image Processing Server (110) is initiated by imaging studies or image studies and provider interpretation (also noted as a report) arriving at the Image Processing Server notes as step 1. After a plurality and combination of engines process the image study, the output is noted as step 2. Studies or images with findings determined to have a high confidence of a potential finding or potential discrepancy with the provider interpretation are injected into the selection for peer review in Step 3. In step A, studies are selected via an engine of engines weighing the value of the high confidence findings and choosing a certain optimized number and type of studies (or images) for physician review. In Step B, the study is evaluated by a physician (who did not make the initial provider interpretation, if applicable). Positive results of that interpretation cause an automatic injection of the study into the peer review system in Step D, which does not occur if the physician finds the study to be negative. In both the positive or negative case, both the results of this interpretation (and any prior interpretations) as well as the Peer Review System interpretation are stored in the database as step C and can be used for future training of engines and engines of engines within the Image Processing Server and Peer Review System. In addition, in Step B, user interaction data is stored.

The workflow further includes a routine first read diagnostic interpretation with blinded peer review engine training workflow loop. During this workflow loop, the Image Processing Server (110) is initiated by imaging studies or image studies and provider interpretation (also noted as a report) arriving at the Image Processing Server notes as step 1. After a plurality and combination of engines process the image study, the output is noted as step 2. Studies or images with findings determined to have a high confidence of a potential finding during the provider primary interpretation are calculated for comparison to the actual physician findings in Step E. In Step B, the study is evaluated by a physician (who did not make the initial provider interpretation, if applicable). In Step C, both the results of this interpretation (and any prior interpretations) as well as the Peer Review System interpretation are stored in the database and can be used for future training of engines and engines of engines within the Image Processing Server and Peer Review System. In addition, in Step B, user interaction data is stored.] FIG. 6B is a block diagram illustrating an example of medical image peer review system according to one embodiment. Referring to FIG. 6B, medical data source 601, in this example, a PACS, sends a set of images to primary review system 602 representing a first set of physicians as primary reviewers. The reviewers of primary review system 602 review and send the findings back to PACS 601. A portion (e.g., 5%) of the images reviewed by the primary reviewers is sent to peer review system 603 representing a second set of reviewers as secondary or peer reviewers. The secondary reviewers review the images to generate a second result that provides second opinion findings. The second result is to validate or verify the primary findings performed by the first set of physicians. The secondary reviewers may perform the review without knowing how the primary reviewers have reviewed the same images, referred to as blind reading. The secondary findings of images can be transmitted back to PACS 601 from peer review system 603.

In addition, according to one embodiment, the medical images reviewed by the primary reviewers are transmitted to image processing server 110. Image processing server 110 will invoke one or more image processing engines 113-115 to independently process the images and generate a third result. Similarly, the image processing engines may perform the review independently without knowing the first result performed by the primary reviewers (e.g., blind reads). The third result may also be utilized to validate or verify the first result.

According to another embodiment, at least a portion of the images (e.g., 5-20%) reviewed by image processing server 110 are transmitted to peer reviewers 603 for further peer review. That is, peer reviewers 603 would receive samples of images reviewed by primary reviewers 602 and samples of images reviewed by image processing server 110. Peer reviewers 603 may review the images received from the primary reviewers 602 and image processing server 110 without knowing the first result produced by the primary reviewers and the third result produced by image processing server 110 (referred to as double blind reads). In one embodiment, image processing server 110 only transmits the images that have been flagged as abnormal to peer reviewers 603 for validation. Similarly, only the images having abnormal findings flagged by primary reviewers 602 may be sent to peer reviewers 603. The findings of primary reviewers 602, image processing server 110, and peer reviewers 603 may be tracked by tracking module 211 and stored as a part of tracking data 504. Alert module 212 may send an alert to a predetermined device such as PACS 601, in response to certain abnormal findings or inconsistent findings amongst primary reviewers 601, engines 502, and peer reviewers 603.

Specifically, according to one embodiment, data source 601, in this example, a PACS, can send studies to first reviewers 602 for review or examination. The studies can include images of any modality, for example, CT, MR, X-Ray, other known or unknown modalities, or any combination thereof. A first set of physicians 601 can review the studies on the workstation and detect/diagnose one or more findings. The findings can be included in the studies or separated from the studies, for example, in a report, email, or 3D medical imaging software. The studies along with the findings by the first set of physicians can be sent from the workstation and stored in the PACS server. The PACS server can send a portion (e.g., between 0% to 100%, more narrowly, between about I % and about 50%, between about 1% and about 25%, between about 1% and about 20%, between about 1% and about 10%) of the total studies between a period of time (e.g., 1 year, 6 months, 3 months, 1 month, 1 week, or 1 day) with the findings by the first set of physicians 602 to peer review system 603 for peer review by a second set of physicians.

In addition, data source 601 can be connected to image processing server 110. The data source 601 can send the total studies with findings by the first set of physicians 602 to the server 110. The image processing server 110 can comprise of one or more engines, one or more e-suites (i.e., combination of one or more engines), or any combination thereof 502. The one or more engines and one or more e-suites can run an algorithm to detect findings. The image processing server 110 can receive and process the total studies with findings by the first set of physicians 602. The image processing server 110 can output machine learned (i.e., trained) possibly higher likelihood of disease studies found with image processing (i.e., flagging studies with findings based on the one or more engines and/or the one or more e-suites). Flagging can mean displaying the actual findings found by the engine/e-suite, the engine/e-suite name, a simple mark representing that the study was processed by the image processing server 110, marking that the study was normal, or any combination thereof. The one or more engines and/or the one or more e-suites can machine learn or be trained such that as the engines/e-suites process more images, the engines/e-suites can detect findings more accurately.

In one embodiment, the image processing server 110 can send a portion of the machine learned possibly higher likelihood of disease studies to peer review system 603. The machine learned possibly higher likelihood of disease studies that are sent to the peer review system 603 can be based on severity of the findings by the engine/e-suite. For example, if the engines 502 within the image processing server 110 flag one study with lung nodules and another study with a minor bone fracture, the image processing server 110 can send the lung nodule study to the peer review system 603 for a second review as the risk to the patient is greater for the lung nodule study compared to the minor bone fracture study. The image processing server 110 can sort studies based on the severity of findings by the engines/e-suites.

The image processing server 110 can be connected to the peer review system 603 such that the machine learned possibly higher likelihood of disease studies found with image processing can be sent to the peer review system 603. The peer review system 603 can receive the peer review samples from the PACS server 601, the machine learned possibly higher likelihood of disease studies from the image processing server 110, or any combination thereof. A second set of physicians can review, reread, blind read, or double-blind read the peer review samples, the machine learned possibly higher likelihood of disease studies, or any combination thereof at the peer review system 603. The peer review samples with the findings from the second set of physicians and the machine learned possibly higher likelihood of disease studies with the findings from the second set of physicians can be sent to the image processing server 110, the workstation, the PACS server, or any combination thereof. In one embodiment, the image processing server 110 can flag studies that are normal. In one embodiment, the image processing server 110 can remove studies that are normal from the peer review samples by a removal engine in the image processing server 110 and/or the peer review system 603.

According to one embodiment, some of the image processing engines 113-115 may individually generate review results and the review results are sent to peer review system 603. The individual results may be integrated by a data integrator of peer review system 603. For example, the PACS server can send the 1,000,000 total studies per year to the workstation (e.g., one or more workstations) for review by the first set of physicians. The first set of physicians can review the studies assigned to them and make findings on each of the studies reviewed. The total studies with the findings by the first set of physicians can be sent to the PACS server. The PACS server can send the peer review samples (i.e., 50,000 studies) with the findings by the first set of physicians to the peer review system 603.

The PACS server can send the 1,000,000 total studies to the image processing server 110. The image processing server 110 can include a COPD e-suite (i.e., a group of engines that can detect COPD based on one or more images, reports, studies, or any combination thereof), a lung nodule engine (i.e., an engine that can detect nodules based on one or more images, reports, studies, or any combination thereof), a bone fracture engine (i.e., an engine that can detect fractures based on one or more images, reports, studies, or any combination thereof), and a lesion engine (i.e., an engine that can detect lesions based on one or more images, reports, studies, or any combination thereof). Each of the engines (i.e., the COPD e-suite, the lung nodule engine, the bone fracture engine, and the lesion engine) can process the 1,000,000 total studies to detect findings. The image processing server 110 can output machine learned possibly higher likelihood of disease studies based on the engines (i.e., studies that have a high likelihood of COPD, lung nodules, bone fractures, lesions, or any combination thereof).

The image processing server 110 can send all or some of the machine learned possibly higher likelihood of disease studies to the peer review system. The image processing server 110 can order or prioritize the machine learned possibly higher likelihood of diseased studies based on, for example, risk to patient health. The image processing server 110 can send the highest risk to patient health studies to the peer review system or another system for review by physicians. The image processing server 110 can display the flagging or hide the flagging on the machine learned possibly higher likelihood of disease studies. The second set of physicians can review the machine learned possibly higher likelihood of disease studies and the peer review samples. The machine learned possibly higher likelihood of disease studies with the findings from the second set of physicians and the peer review samples with the findings from the second set of physicians can be sent to the image processing server 110 or to the PACS server.

The machine learned possibly higher likelihood of disease studies can be studies where there is a high probability that there are findings. The machine learned possibly higher likelihood of disease studies can be studies where there is a high likelihood that the initial review by the first set of physicians may have misdiagnosed the study. If multiple engines flag one study as part of the machine learned possibly higher likelihood of disease studies, such a study can be included in the machine learned possibly higher likelihood of disease studies once (i.e., no duplicate studies can be included).

The operations and results of the reviewers (e.g., primary reviewers, image processing engines, and peer reviewers) can be tracked by tracking module 211. The tracking engine 211 can track information including which studies have been received by image processing server 110, which studies have been processed by which engines, which studies have been flagged by which engines, which studies have been flagged by which engines, which studies have been sent as part of the peer review samples, engine name, findings from the engine, normal studies, severity of findings, severity ratings, rankings of studies based on findings, severity, and/or number of findings, or any combination thereof.

The tracking engine 211 can track findings based on the engines for each study. The tracking engine 211 can store such information in the memory of the image processing server 110. The peer review samples and all or some of the machine learned possibly higher likelihood of disease studies can be sent to the peer review system 603. A combined sample engine of peer review system 603 (not shown) can randomly mix the peer review samples and the machine learned possibly higher likelihood of disease studies to form combined samples such that the second set of physicians may not be able to determine whether the study being reviewed is from the peer review samples or the machine learned possibly higher likelihood of disease studies. The combined sample engine can place the machine learned possibly higher likelihood of disease studies and the peer review samples together in a pre-determined order based on findings, disease, engine name, risk to health, physician, date of study, patient, random, or any combination thereof.

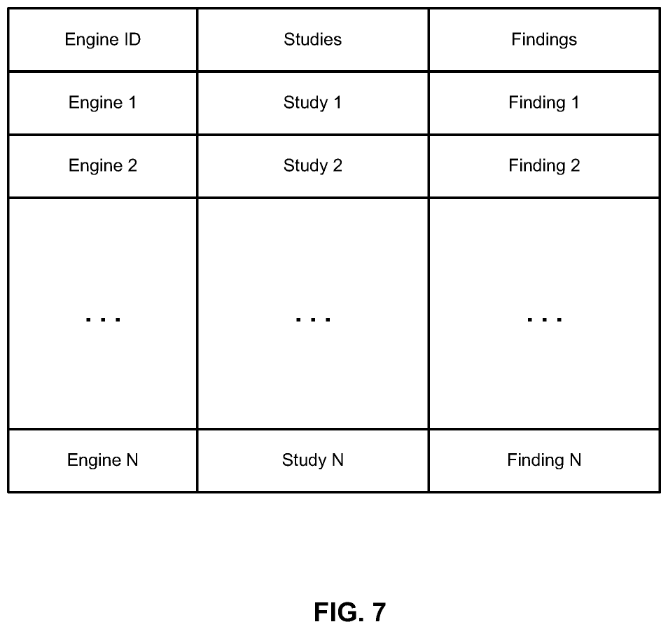

For example, as shown in FIG. 7 as an example of a tracking table storing the tracking data, the tracking engine 211 can correlate that a first engine can output (i.e., flag) a first study and the first study can have a first finding. The tracking engine 211 can correlate that a second engine can output (i.e., flag) a second study and the second study can have a second finding. In one embodiment, the machine learned possibly higher likelihood of disease studies can include studies from the peer review samples. The image processing server 110 and/or the peer review system can remove duplicate studies from the combined samples. In one embodiment, the image processing server 110 and/or the peer review system 603 can remove normal studies (i.e., studies with no findings) from the combined samples and/or peer review samples. The tracking engine 211 can track which engines were removed from combined samples and/or peer review samples.

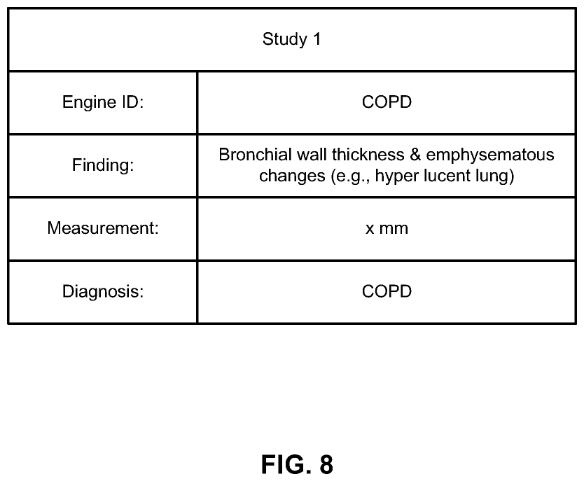

According to one embodiment, a report can be generated by report module 214 based on the tracking data and the report can be transmitted to a requested destination such as a particular user or system. FIG. 8 is a block diagram illustrating an example of a report of findings of a particular study according to one embodiment. The report includes data indicating which image processing engine has been used; what findings have been identified; the measurement of the findings; and the diagnosis result.

According to one embodiment, features from each processing engine on image data will need to correspond to elements in a report. An NLP system or an NLP module may be utilized to parse the text within a report to match the extracted image features. A set of NLP rules may be define to determine which processing engines to run the NLP algorithm. For example, if a hand x-ray states “no acute fracture or traumatic subluxation,” a hand x-ray fracture algorithm is initiated via the image processing server to conform the report accuracy.

According to one embodiment, referring back to FIG. 6B, the system can be utilized for double blind reads to validate findings. For example, the PACS server 601 can send the total studies to review system 602 for review by the first set of physicians to determine findings for each of the total studies. The review system 602 can send the total studies with findings by the first set of physicians to the PACS server 601. The PACS server 601 can send the total studies with the findings to the image processing server 110. First engine 113, second engine 114, and third engine 115 of image processing server 110 can process each of the total studies with or without the findings by the first physician. The image processing server 110 can output the machine learned possibly higher likelihood disease studies with no claims/findings generated by the engines 113-115 displayed in each of the machine learned possibly higher likelihood disease studies (i.e., the second set of physicians would not know whether the study being reviewed was flagged and/or processed by image processing server 110).

The machine learned possibly higher likelihood disease studies and peer review samples can be randomly combined in the peer review system 603 to form the combined samples. The combined samples can be reviewed by the second set of physicians. The second set of physicians can validate the findings of engines 113-115 and the first set of physicians. In other words, engines 113-115 can be validated by a double-blind read by comparing the findings from the first set of physicians and the findings by engines 113-115; and comparing the findings by the second set of physicians and the findings by engines 113-115.

For example, the first physician can review a first study and determine that the images in the first study contained bone fractures. The first study can be processed by a bone fracture engine. The bone fracture engine can flag the first study as having bone fractures without displaying any claims/findings in the first study such that the second physician would not know which engine was used or what findings were detected by the bone fracture engine. The second physician can review the first study and confirm that the first study has bone fractures. Here, the bone fracture engine flagged the first study as having bone fractures which was validated by the first physician's finding and the second physician's finding.

In another example, a first physician can review a first study and determine that the images in the first study did not contain lung nodules. The first study can be processed by a first lung nodule engine. The first lung nodule engine can flag the first study as having lung nodules without displaying any claims/findings in the first study such that a second physician would not know which engine was used or what findings were detected by the first lung nodule engine. The second physician can review the first study and confirm that the first study has lung nodules. Here, the first lung nodule engine flagged the first study as having lung nodules which was validated by the second physician but misdiagnosed by the first physician.

According to another embodiment, a first physician can review a first study and determine that the first study comprises a first finding. The first study can be processed by a first engine in the image processing server 110. The first engine can flag the first study as having the first finding and output the first study as the machine learned possibly higher likelihood of disease studies without indicating any claims/findings on or with the first study. The second physician can review the first study and confirm that the first study has the first finding. The tracking engine can track the first study findings of the first physician, the second physician, the first engine, or any combination thereof. A comparator engine (not shown) can compare the findings of the first physician, the second physician, the first engine, or any combination thereof to determine if the first engine can be validated via a double-blinded read. The tracking engine can keep track of whether operations of the first engine was validated or invalidated.

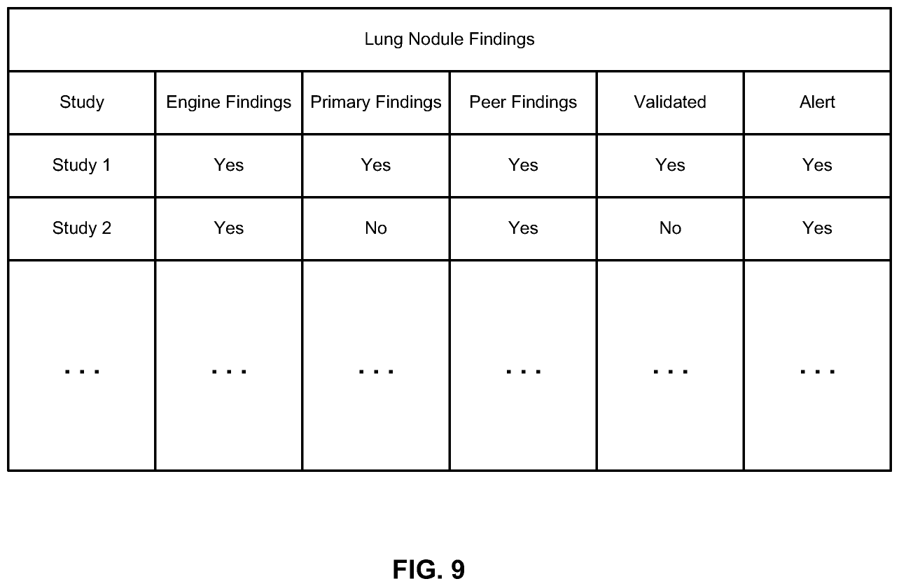

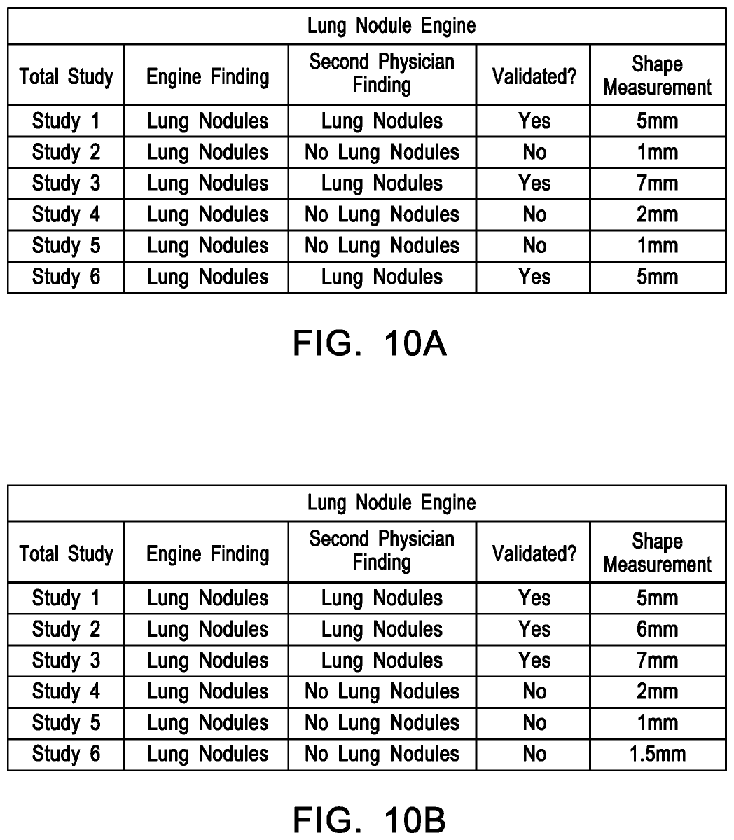

For example, a first physician can review a first study and determine that a first study contains lung nodules. The first study can be processed by a first lung nodule engine. The first lung nodule engine can flag the first study as having lung nodules and output the first study as the machine learned possibly higher likelihood of disease studies without indicating any findings/claims. The second physician can review the first study and confirm that the first study has lung nodules. The tracking engine can track findings of the first physician, the second physician, the lung nodule engine, or any combination thereof (as a part of tracking data as shown in FIG. 9). A comparator engine can compare the findings of the first physician, the second physician, the lung nodule engine, or any combination thereof, to determine whether the lung nodule engine can be validated via the double-blinded study.

The lung nodules engine can be validated because the first physician finding, the second physician finding, and the lung nodule engine finding were consistent/equal, as shown in FIG. 9. Whether the engine was validated or not can be displayed on the client device (e.g., a workstation, web site, mobile device or any future computer i/o device), the peer review system 603, the image processing server 110, engine profile, the Peer Review medical imaging software, or any combination thereof, and such results can be stored in the image processing server 110.

In one embodiment, when a comparator engine (not shown) compares the findings of the first physician, the second physician, and the first engine, the comparator engine can output statistics based on the similarities of the findings between the first physician, the second physician, and the first engine. Such information can be stored in the memory and/or persistent storage device. For example, the comparator engine can display that the first engine had 80% correct findings with the first physician; and the first engine had 20% correct findings with the second physician.

According to another embodiment, the tracking engine can track the first engine data (e.g., which studies have been sent to the image processing server 110; which studies have been processed by which engines; which studies have been flagged by which engines; which studies have been flagged by multiple engines; which studies have been sent as part of the peer review samples; engine name; findings; data on validated and/or invalidated machine learned (in all cases this may be alternatively a deterministic or other non-machine learning computational method) possibly higher likelihood of disease studies; data on features of the images in the studies including, but not limited to, wall thickness, texture, slope, measurements, similarities, anatomic identifiers or any combination thereof; user interactions of the first physician and the second physician; time for diagnosis; or any combination thereof). Based on the first engine data, the training engine can train the first engine with machine learning with supervised or unsupervised methodology, or manual adjustment of a common deterministic or statistical engine. Such feedback and analyses can improve the algorithm of the first engine. Such machine learning can improve the selection of the machine learned possibly higher likelihood of disease studies. Such machine learning process based on the first engine data can be iterative, automatic, or manual.

For example, after the total studies are run through a lung nodule engine, the machine learned possibly higher likelihood of lung nodule studies can be flagged without displaying any claims/findings on the studies. The machine learned possibly higher likelihood of lung nodule studies can be injected into the workflow of the current peer review system in use, and thereby will be reviewed by the second physician to validate or invalidate the findings of the lung nodule engine without the physician having prior knowledge of the findings. The training engine (e.g., machine learning engine) can train the lung nodule engine based on the lung nodule engine data that are recorded by the tracking engine. For example, the training engine can train the lung nodule engine based on validation/invalidation data, features of the images in the studies resulting in validation/invalidation such as shape measurement (as shown in FIGS. 1OA and IOB), texture, sphericity, other measurements, or any combination thereof.

Using machine learning techniques, the actual feature(s) that create the findings are not always known because neural network technology uses inferences and similarities which are no formulas. The training engine can train the lung nodule engine based on the lung nodule engine data from the second physician (as shown in FIG. 1OA), the first physician (as shown in FIG. 1OB), studies with known findings, or any combination thereof. The training can occur on the GUI of the image processing server 110 or within the Peer Review System platform software application. As shown in FIGS. 1OA-1OB, when the findings between a physician and an image processing engine are consistent, the findings are validated. If the findings are inconsistent, they will be invalidated.

According to one embodiment, the peer review sessions present the unique opportunity to inject studies to be read that have either a very high or very low statistical confidence of having conditions or indications that are symptomatic of certain patient ailments. This confirmation can be used as the ground truth needed to confirm or reject findings by engines in a purely blinded fashion, since the physician will not be privy to the reason(s) that the images were injected for re-reading. The final imaging report can be computer read looking for confirmation of the suspected patient conditions. Alternatively, the system may inquire to the physician by way of a selection box or other means to allow the direct feedback to be gathered at the end of the interpretation as to whether the high or low probability conditions do or do not exist. Alternatively, this feedback may be gained by observing interactions with the Peer Review System during diagnostic interpretation.

High or low probability conditions and/or findings can be detected by trained engines or algorithms which are based on deterministic or machine learned (or deep learned) tests, in some cases applied to medical images and called computer vision. This science and the creation of algorithms relies upon the availability of a large number of labelled image data sets in order to train or test the algorithms, or engines, that can subsequently find or create similar findings similar in some way relating to those that it was trained on. As these additional peer review (high or low test probability) studies are read by a professional in the normal course of peer review (blinded or unblended reading by a second professional and adjudication by a third in the case of disagreement) the results in these processed data are validated via similar findings, or rejected based on discordant results. This happens similarly again for discordant results. This feedback is returned to the engine execution and training system (also known as the platform) and is used to re-train or further train the algorithm, either automatically (unsupervised) or based upon engine-author or owner preferences and guidance (supervised). Not all feedback will create a more intelligent engine, as such there are various benefits to an unsupervised or supervised (governed) training approach.

Unsupervised engine learning can be accomplished using an engine that runs in supervised or unsupervised mode to select which engines are run for various circumstances to produce the most valuable results. For example, if there are 100 engines that can run on 10,000 image data sets (or numerical/other data, if not images) for a given day's work output, then the “engine of engines” would select which engines run on which of the applicable data and experiment to achieve the highest possible value for the day (based on the assigned value of each finding). This value represents the value of the findings that were actually confirmed when the study was re-read by a physician in the peer review process.

User inputs or mapping of values to each type of finding made for each engine, including means to set defaults for new findings that were not specifically set already. Physician engagement and interactions to adjust, accept, reject, cancel or add findings of the engines such as indicator points (look here for something without naming it or look for something with a specific name/type)

Ability for an engine to assimilate the end user interaction feedback and the underlying data in order to establish these data as future engine training data, based on any of the following: access security controls, cloud access controls, governance features, training features type of feedback, end user characteristics, variance in measured engine performance with and without the inclusion of any, all or part of the end user interaction feedback. Choosing the proper algorithms to return the highest activities being organized into categories (for example by procedure code or CPT code, procedure type, body part, imaging modality and other factors relevant to patient care and healthcare provider workflow). Files contain the automated values, are uploaded into a system and restored, edited and saved. Values in these files are stored, for example in XML, retained in files, restored to files, and stored with versioning of all data extracted into a database that is capable of delivering back searches of values, comparisons and trending over time, and the ability to run statistics to deliver back a mean, average or other statistical compilation of values that can be reconstructed back into a file format able to be returned to the diagnostic or viewing system.

In one embodiment, upon a request (e.g., a Web request such as JSON request), a document obtained from the request is converted into a filesystem. In the transformation, each key-value-pair in the request document corresponds to a file or directory in a folder on the peer review system. Key-value-pairs where the value is of type Boolean, number, or string are transformed into to files named by the key and containing the value. Key-value-pairs where the value is an array type are transformed into directories named by the key and containing a files or directories named by their index in the array with contents following these same transformation rules. Key-value-pairs where the value is a nested document are transformed into directories named by the key and containing a filesystem following by these same transformation rules.

The newly created ‘input’ filesystem is bound to the executable software container (e.g. Docker) along with a writable ‘output’ filesystem to hold the container's output. When the container image is done executing the ‘output’ filesystem is then transformed into a response document (e.g., JSON document) using the inverse of the transformation rules. Additionally, creating a small companion container image to be paired with each executable container to facilitate the input and output transformations in such a way that is compatible with existing container load balancers and schedulers. This will require the companion container (docker) image to use the remote container (docker) API as well as the NvidiaGPUlnfo service to connect to its own host to then run sibling the executable container (docker) image.

This peer review system with its features for matching engine outputs and/or findings with those that have physician agreement, utilization and appreciation provides an automated means to collect validation data for compliance with regulatory bodies such as CE mark and FDA 510k or PMA filings, or even the means to collect the physician validation and/or ground truthing to support new such filings. Regulatory bodies employ a systematic and well documented approach to data collection that is supported as part of the peer review process and the engines that are developed or validated by such means are identical to those that are used in clinical practice outside of the peer review process. In one embodiment, the peer review system serves a dual purpose to ensure the data collection requirements, including images and clinical content as well as physician verification support, for the regulatory and quality assurance requirements of each engine are met in whole or in part. Such support is also provided for a combination of engines, or engine of engines.

In addition to such clinical verification, the peer review system supports the collection and compilation of documentation necessary for marketing an engine and/or gaining approval for its diagnostic use as a medical device, which varies based upon the specific requirements of each country. One component of the peer review system validation and compliance documentation is a technical file, which tracks changes and updates to software. The technical file also contains the engine author's representations about the specific implementation of a given medical device including compute environment and server requirements, as well as clinical capabilities and feature specifications. This may also include specifications of how the medical device is implemented within a given system. In one embodiment, the peer review system database with its associated or included engine version tracking and image/content cohort version tracking, as well as the physician feedback and utilization data, serves as a means to comply with these regulatory and quality system requirements in an automated fashion, allowing for near complete or fully complete creation of the technical and clinical validation and verification sections of regulatory and quality assurance submission(s).

In order to ensure regulatory compliance with engines learned from third party data and or companies, the system tracks regulatory information in the course of its ordinary use for peer review. The peer review system contains governance of these engines by algorithm developers (authors) and/or companies in order to manage the quality of engines in use. The system selects a peer review cohort of studies from the stream of images being read or interpreted clinically based on the peer review engines selected and the setting chosen based on peer review system settings. The system provides the capability to import previously labelled data in the form of a saved state image set or sets and/or image data or clinical content cohort(s). The system is used to create the image and clinical content cohorts for clinical regulatory validation demonstrating measured agreement or disagreement with each finding in the cohort as compared to a known-good reference cohort (often from another engine, product or third party, or from the assembly of expert read cohorts assumed to be ground truthed).

The results of the new non-regulatory approved set of engine outputs is compared to the regulatory approved set of engine outputs and this agreement or disagreement is presented to the physician for review and acceptance with all such results documented it the peer review system. All final confirmation, rejection or adjustment by the physician or clinician is documented in the peer review database. The peer review system is used to demonstrate that the engine(s) being approved operated with satisfactory results, workflow and safety, and/or similar sensitivity and specificity, with measured statistical agreement confidence as can be achieved through the application of the standard peer review features of the platform.

An algorithm developer for an engine can be a physician, company, software engineer, data scientist, or anyone with access to data to build engines. An engine may also be a derivative work that is the output of many other engines working in a supervised or unsupervised fashion. The system of governance is implemented in the peer review system to manage engines developed to be used in the platform and assign access rights to be able to run, manage, version, track usage, control output, grant access to others, publish the engine for use, restrict it for private or limited use, upload documentation, verification and validation data as well as marketing materials and proof of quality, regulatory clearance status and documentation, specifics about usage and customer satisfaction, and other forms of related clinical content and information. Investigational use and clinical use engines are differentiated by medical claims made by engine authors that have different governance, pricing, performance guarantees, legal contracting for use, and other factors ensuring that such engines are suitable for marketing and use as medical devices.

Along with automatically generated reports for regulatory filing, such as CE mark, 51Ok, or PMA; the governance features for managers will perform automated post market surveillance by looking at usage data and feedback data. Usage data can be how the algorithm was used in clinical practice. Feedback data may also include clinical outcomes data associated with the clinical image data set and clinical content being assessed and measured. It may be Boolean or enum selections, or other clinical observations, at the point of interpretation or after interpretation by of a physician, either before or after processing by the peer review system regarding performance or prospective/retrospective analysis of downstream clinical implications of the engine results, and ensemble of engines (combination) returning results, or those of an engine of engines.

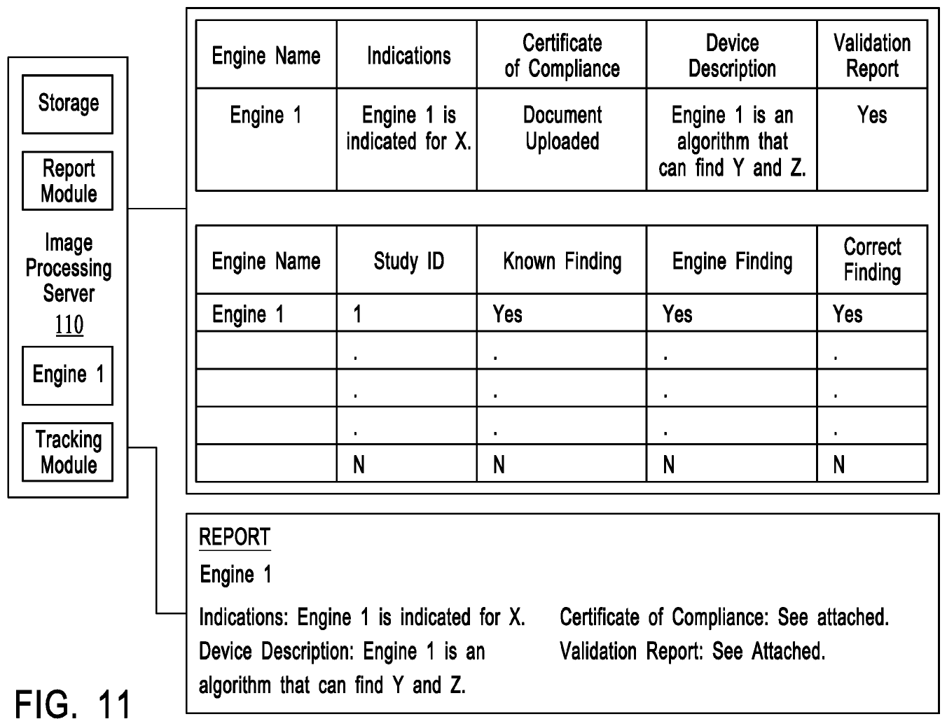

FIG. 11 illustrates a report generation process according to one embodiment. Referring to FIG. 11, a first user can upload or complete user inputted information. The user inputted information can be associated with the engine and stored in storage. The user can upload documents associated with the first engine via a client application. The user uploaded documents can be associated with the first engine and stored in storage. For example, the user can input information or upload documents related to a medical device user fee coversheet, a Center for Devices and Radiological Health (CDRH) premarket review submission, certificate of compliance, table of contents, cover letter, indications for use, 510k summary, truthful and accuracy statement, class III certification summary, name of the device/engine, description of the device/engine, predicate, comparison of the device/engine with the predicate, intended use, proposed labeling, expiration, or any other information or combination thereof. Such information can be stored in the storage of the image processing server or the application store. Such information can be associated with the first engine. Such information or any subset of the information, if requested by the first user or if part of the application template, can be taken from storage and can be included in the report. If any information from a template is missing, a prompt may be generated to notify the user that the user is missing information and can include the information.

The user can request a report for the first engine from a client application on a client device or webpage. When the user requests a report from the application, the application can send a request to the image processing server via a network. The report module can compile the user inputted information, tracking information, study information, validation information, uploaded documents, other information inside its database or information or systems to which it is integrated, or any combination thereof for the first engine, cohort or constrained data inquiry from storage and compile a report. The report can be preconfigured using a report template such that the report is automatically completed based on the stored information. The report can be based on user specifications of which data the user needs from the storage of the image processing server by selecting specific fields on the application. For example, the user may only want the validation and the indications for use. The report can be sent to the client application or website. The report can be manipulated by the user once sent to the application or can be manipulated by the user before the report is sent to the application.

For example, the user can include additional information, update information, remove information, or any combination thereof. The Application can comprise of predetermined report templates where the report comprises of all information for 5IOK submissions, PMA submissions, Clinical Trial submissions, Insurance related submissions, or any other submissions requiring validation of an engine. One or more users from one or more medical institutes can review the report to verify the results of the report via the application. Such reports can be auto generated when the user requests such report via the application. The application can allow the one or more users to mark (e.g., flag, mark red/green, circle issues on each study, or any combination thereof) whether the report is verified or mark specific studies where the results may be incorrect. In another embodiment, an adjudicating group can determine whether the engine is valid or not via the application. Closed loop machine learning produces clinical trial validation data sets.

For example, a user can upload an engine (e.g., lung nodule) to the cloud. The user can machine learn/train the lung nodule engine with studies via the application. If access is granted to the lung nodule engine to other users, other users locally or via the cloud can train/machine learn the lung nodule engine with studies. When the lung nodule engine is optimized, the user can run the lung nodule engine on one or more studies with known or unknown findings. The tracking nodule can track information such as study ID, known findings, unknown findings, lung nodule engine findings, accuracy of lung nodule engine, percent accuracy of lung nodule engine, or any combination thereof.

The user can also input other lung nodule engine information via the GUI of the application such as intended use, indications for use, description which can be associated with the lung nodule engine via the tracking module and stored in storage. The user can request a report from the application, for example a 5 IOK report. The report module can receive the request for the 51Ok report for the lung nodule engine and complete the applicable fields in the 51Ok report template based on information stored in storage associated with the lung nodule engine. The 51Ok report for the lung nodule engine can then be sent to the application so that the user can view the 510k report, update the 510K report, download the report, print the report, submit the report directly to a regulatory authority, or any combination thereof.

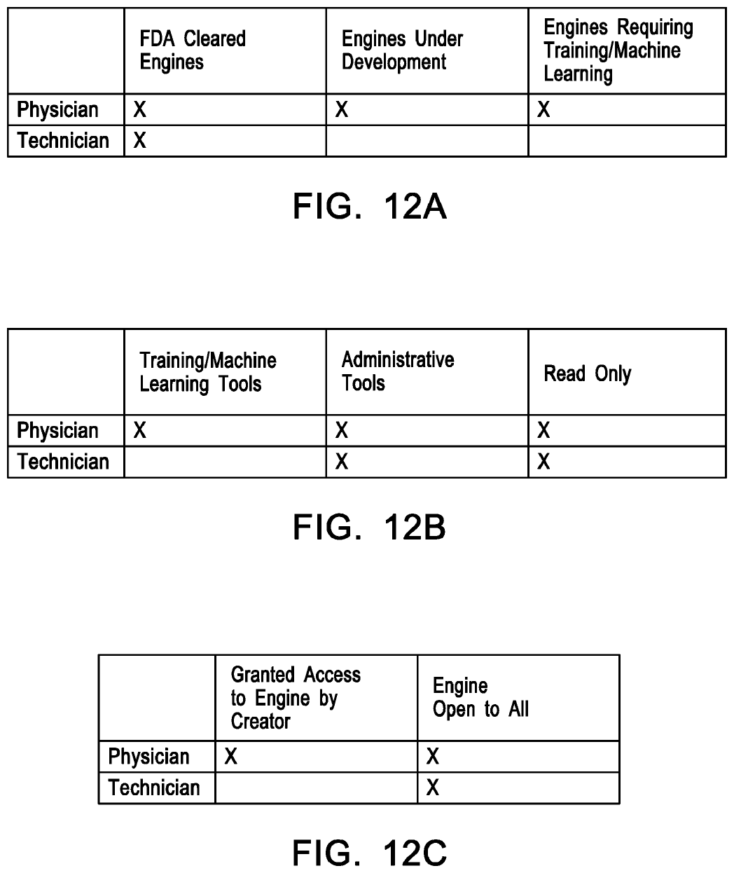

FIGS. 12A-12C illustrate an access control table for authenticating users for image processing according to one embodiment. A client application can have access control such that certain users are given certain user privileges. For example, a physician can have access to and use any engine available to the physician (i.e., if the physician was granted access to the engine by the creator) on the cloud for clinical and non-clinical use. For example, a non-physician can have access to and use any engine available to the non-physician on the cloud for non-clinical use and as long as they were granted access to those engines by the creators. For example, the creator of the engine can give access to another physician to use the engine and/or machine learn/train the engine. In another example, the creator can give access to a physician or a group of physicians from one or more medical institutes to process studies through the engine.

According to one embodiment, image processing server 110 further includes an access control system to control access of engines, resources (e.g., image processing tools) and/or medical data stored in a medical data store. Users may or may not access specific engines, certain portions of resources and/or medical data stored in medical data store dependent upon their respective access privileges. The access privileges may be determined or configured based on a set of role-based rules or policies, as shown in FIGS. 12A-12C. For example, physicians can only access certain engines such as FDA cleared engines, engines under development, engines requiring training, or their own uploaded engines and data, or any combination thereof. Some users with certain roles or credentials can only access some of the tools provided by the system, as shown in FIG. 12B. Some users with certain roles can only perform certain steps or stages of the training/machine learning. Steps or stages are incorporated into the tools and might include identifying and/or measuring instructions or validation/acceptance of feedback from previously performed steps or stages. Some users with certain roles are limited to certain types of processes.

In one embodiment, the access control system can control access based on Health Insurance Portability and Accountability Act (HIPAA) compliance. For example, a first physician can grant access to a second physician to medical image data and/or engines. The first physician can grant access to the engine and can also request/send a Business Associate Agreement (BAA) via the image processing server to ensure compliance with HIPAA requirements. The second physician can access an engine after the second physician agrees to the BAA. The BAA can be tracked on the user profile of the first physician and the second physician. The application can have an option to anonymize protected health information on studies.

Note that the rules or policies as shown in FIGS. 12A-12C are described for the purpose of illustration only; other rules and formats may also be applied. According to some embodiments, access levels can be configured based on a variety of parameters, for example, types of engines, whether engines have been approved by FDA, whether engines are still being developed, whether the engine has been validated, whether the engines needs to be further trained, types of tools or steps within a tool, functions (e.g., uploading, downloading, viewing, manipulating, auditing, validating, etc.), ability to give others access (e.g., second opinion, referrals, experts, family, friend etc.), patients, engine (e.g., may only have access to certain number or uses of engines per month, for example, dependent upon a licensing agreement), medical institution, specialty, reimbursement or billing code (e.g., may only have access to perform certain procedures that are reimbursed by insurance), admin access level, clinical trial or research project, way of viewing data, HIPAA clearance, or any combination thereof.

Similar access controls may be provided for image data cohorts, clinical data cohorts, feedback and data in the database or contained within the Peer Review System. The cloud model allows for physicians or other participants all over the world to participate in using the application. The local model allows for doctors or other participants within one network or within a department to participate in using the application without patient information leaving their environment. The access control of the application can control what engines and tools are used and how and by whom.

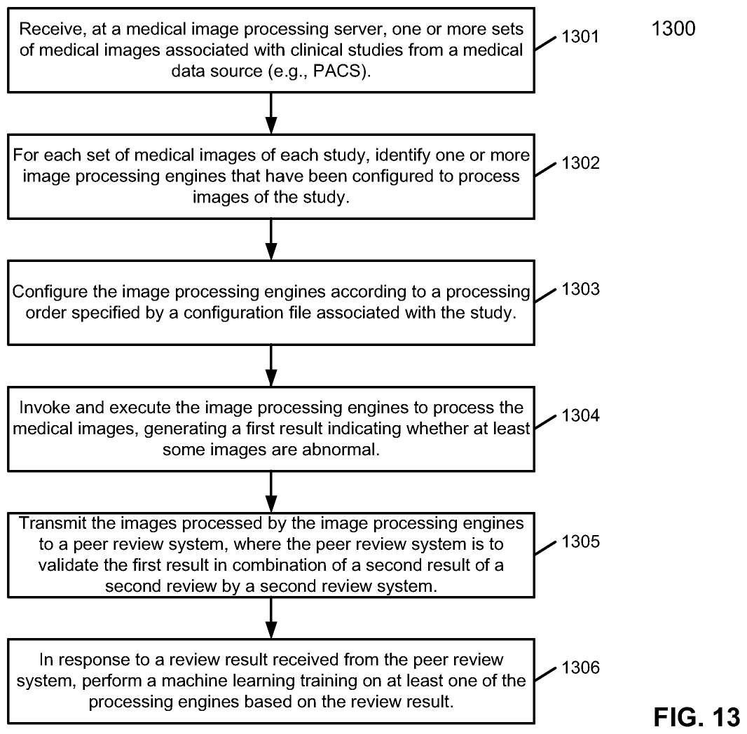

FIG. 13 is a flow diagram illustrating a process of processing medical images according to one embodiment of the invention. Process 1300 may be performed by processing logic which may include software, hardware, or a combination thereof. For example, process 1300 may be performed by image processing server 110. Referring to FIG. 13, at block 1301, processing logic receives sets of medical images associated with a set of clinical studies from a medical data source such as a PACS. At block 1302, for each set of medical images of each clinical study, processing logic identifies one or more image processing engines that has been configured to process images of the clinical study. At block 1303, processing logic configures the image processing engines according to a processing order specified by a configuration file associated with the clinical study. At block 1304, processing logic invokes and executes the image processing engines to process the medical images, generating a first result. The first result includes information indicating abnormal medical images. At block 1305, processing logic transmits the abnormal medical images to a first review system. The first review system is to validate the first result in combination of a second result of a second review performed by a second review system. At block 1306, in response a review result received from the peer review system, processing logic may perform a machine learning operation to train at least one of the processing engines to modify one or more processing algorithm to improve future image processing operations (e.g., efficiency, accuracy).

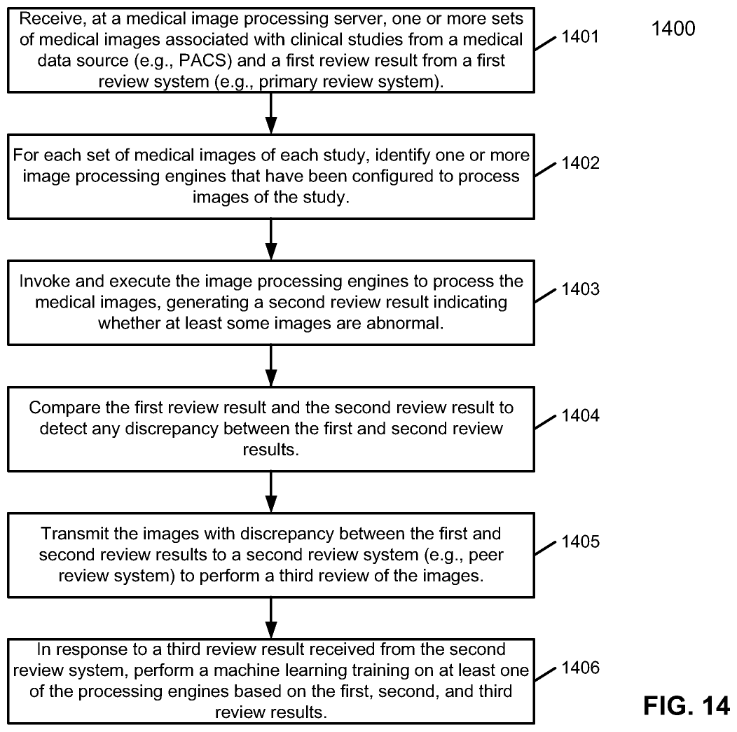

FIG. 14 is a flow diagram illustrating a process of processing medical images according to another embodiment of the invention. Process 1400 may be performed by processing logic which may include software, hardware, or a combination thereof. For example, process 1400 may be performed by image processing server 110. Referring to FIG. 14, at block 1401, processing logic receives sets of medical images associated with a set of clinical studies from a medical data source such as a PACS, as well as a first review result received from a first review system (e.g., a first peer review system or a primary review system). At block 1402, for each set of medical images of each clinical study, processing logic identifies one or more image processing engines that has been configured to process images of the clinical study. At block 1403, processing logic invokes and executes the image processing engines to process the medical images, generating a second review result. The second review result includes information indicating abnormal medical images if there is any. At block 1404, processing logic compares the first review result and the second review result to detect any discrepancy between the first and second review results. At block 1405, processing logic transmits the medical images with discrepancy (e.g., conflict between the first and second review results) to a second review system (e.g., a second peer review system). The second review system is to perform another review on the conflicting medical images, generating a third review result. At block 1406, in response the third review result received from the second review system, processing logic may perform a machine learning operation to train at least one of the processing engines to modify one or more processing algorithm to improve future image processing operations (e.g., efficiency, accuracy).

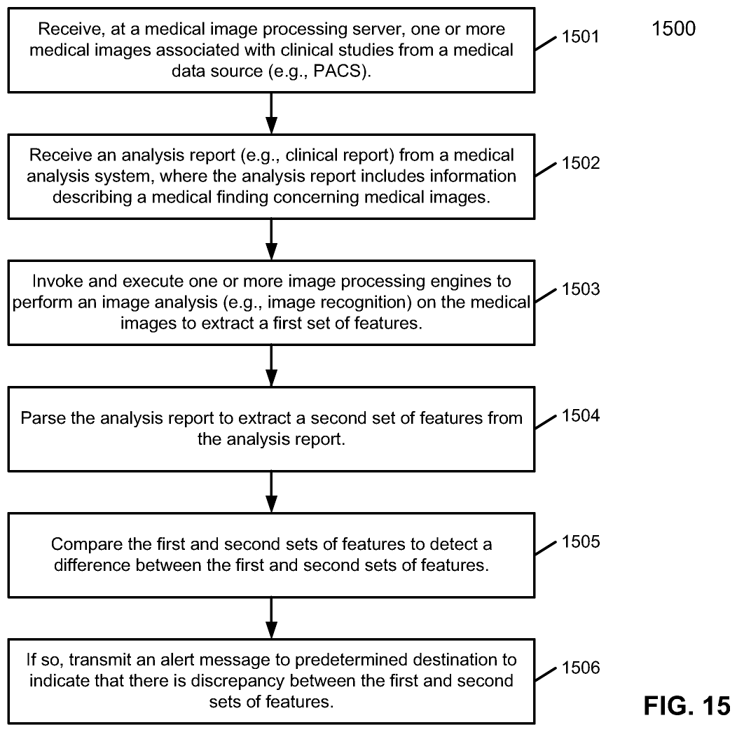

FIG. 15 is a flow diagram illustrating a process of processing medical images according to another embodiment of the invention. Process 1500 may be performed by processing logic which may include software, hardware, or a combination thereof. For example, process 1500 may be performed by image processing server 110. Referring to FIG. 15, at block 1501, processing logic receives a set of one or more medical images from a medical data source such as a PACS system. The medical images may be associated with a clinical study or a patient. At block 1502, processing logic receives an analysis report from an analysis system. The analysis report includes information describing a medical finding of the medical images. For example, the analysis report may be a clinical report produced by a physician or automatically generated by a computing system. At block 1503, processing logic invokes one or more medical image processing engines to perform an image analysis such as an image recognition on the medical images to extract a first set of features from the medical images.

At block 1504, processing logic extracts a second set of features from the analysis report. The extracted features may indicate a medical finding or estimation of the medical images. At block 1505, processing logic compares the first set of features and the second set of features to detect whether there is any difference between the two. If so, at block 1506, processing logic transmits an alert message to a predetermined destination (e.g., an administrator system, the physician who generated the analysis report, or another physician who can perform a peer review). The alert message may indicate that someone needs to follow up with the patient or the medical images. According to one embodiment, the medical images may be transmitted to a peer review system to allow a peer reviewer to perform a peer review to further validate or invalidate the medical finding of the analysis report and/or the image analysis performed by the medical processing engines.

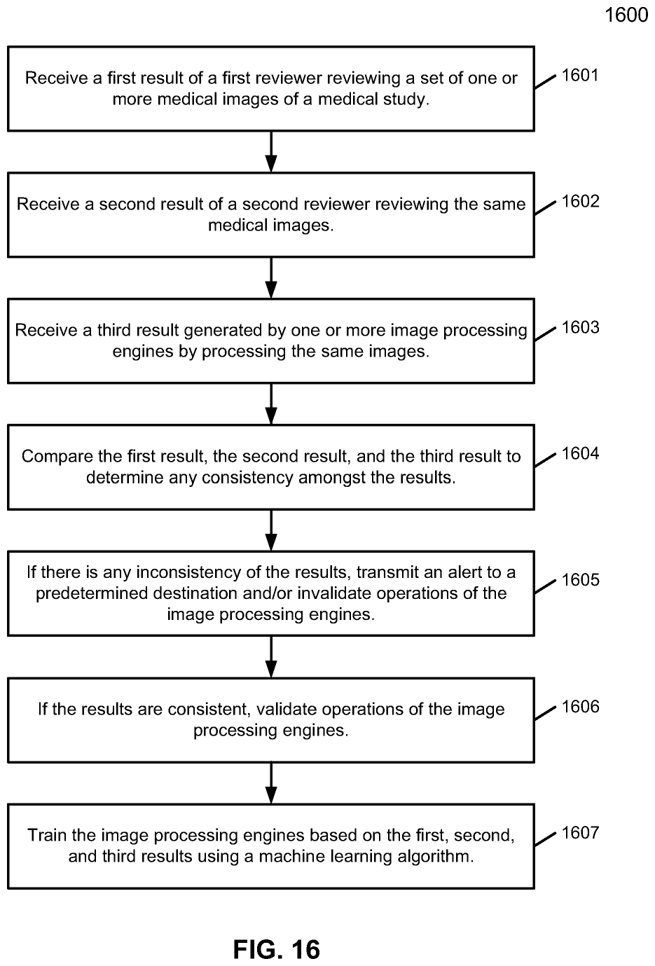

FIG. 16, is a flow diagram illustrating a process of processing medical images according to another embodiment of the invention. Process 1600 may be performed by processing logic which may include software, hardware, or a combination thereof. For example, process 1600 may be performed by image processing server 110. Referring to FIG. 16, at block 1601, processing logic receives a first result of a first reviewer reviewing one or more medical images of a clinical study. At block 1602, processing logic receives a second result of a second reviewer reviewing the same images independently. At block 1603, processing logic receives a third result generated by one or more image processing engines processing the same medical images. At block 1604, processing logic compares the first, the second, and the third results to determine any inconsistency amongst the results. If there is any inconsistency of the results, at block 1605, processing logic transmits an alert to a predetermined destination and/or invalidate operations of the image processing engines. If the results are consistent with each other, at block 1606, processing logic validate the operations of the image processing engines. At block 1607, the image processing engines are trained based on the results using a machine learning algorithm. This method provides a means to achieve improved sensitivity and specificity by combining the outputs of engines from authors who may not even know each other but have granted access to their engines.

According to certain embodiments, a variety of image processing tools can be accessed by a user using the diagnostic image processing features of the Peer Review System. Alternatively, such image processing tools can be implemented as image processing engines 113-115 which are then evoked in other third party systems, such as a PACS or EMR, or other clinical or information system. The following are examples of medical image processing tools present in a current leading semi-automated image viewing and advanced visualization system that may be included and/or further automated, or converted to engines, as part of the image processing system described above. These examples are provided for illustrative purposes and not intended to be a limitation of the present invention.

Vessel Analysis tools may include a comprehensive vascular analysis package for CT and MR angiography capable of a broad range of vascular analysis tasks, from coronary arteries to aortic endograft planning and more general vascular review, including carotid and renal arteries. Auto-centerline extraction, straightened view, diameter and length measurements, CPR and axial renderings, and Vessel Track mode for automated thin-slab MIP may be included.

Calcium scoring tools may include identification of coronary calcium with Agatston, volume and mineral mass algorithms. An integrated reporting package with customization options may be included.

Time-dependent analysis (TDA) tools may include time-resolved planar or volumetric 4D brain perfusion examinations acquired with CT or MR. The TDA tools may support color or mapping of various parameters such as mean enhancement time and enhancement integral, with semi-automated selection of input function and baseline, to speed analysis. TDA tools may support rapid automated processing of dynamic 4D area-detector CT examinations to ensure interpretation within minutes of acquisition.

CT/CTA (Computed tomography angiography) subtraction tools are used in the removal of non-enhancing structures (e.g. bone) from CT angiography examinations, the CT/CTA option includes automatic registration of pre- and post-contrast images, followed by a dense-voxel masking algorithm which removes high-intensity structures (like bone and surgical clips) from the CTA scan without increasing noise, aiding with the isolation of contrast-enhanced vascular structures.

Lobular decomposition tools identify tree-like structures within a volume of interest, e.g. a scan region containing a vascular bed, or an organ such as the liver. The LD tool can then identifies sub-volumes of interest based on proximity to a given branch of the tree or one of its sub-branches. Research applications include the analysis of the lobular structure of organs.

General Enhancement & Noise Treatment with Low Exposure tools may include an advanced volumetric filter architecture applying noise management techniques to improve the effectiveness of 3D, centerline, and contouring and segmentation algorithms even when source image quality is not optimum.

The Spherefinder tools perform automated analysis of volumetric examinations to identify the location of structures with a high sphericity index (characteristics exhibited by many nodules and polyps). Spherefinder is often used with Lung or Colon CT scans to identify potential areas of interest.

Segmentation, analysis & tracking tools support analysis and characterization of masses and structures, such as solitary pulmonary nodules or other potential lesions. Tools may identify and segment regions of interest, and then apply measurement criteria, such as RECIST and WHO, leading to tabulated reporting of findings and follow-up comparison. Display and management of candidate markers from optional detection engines may be supported, including Spherefinder.

Time volume analysis tools may provide automated calculation of ejection fraction from a chamber in rhythmic motion, such as a cardiac ventricle. A fast and efficient workflow may be included to enable the user to identify the wall boundaries of interest (e.g. epicardium and endocardium) and, based on these user-confirmed regions of interest, to report ejection fraction, wall volume (mass) and wall thickening from multi-phasic CT data. Tabulated reporting output is included.

Maxillo-facial tools support the analysis and visualization of CT examinations of the Maxillo-facial region, these tools apply the CPR tool to generate “panoramic” projections in various planes and of various thicknesses, and cross-sectional MPR views at set increments along the defined curve plane.

Applicable to endoluminal CT or MR investigations such as colon, lungs, or blood vessels, the Flythrough tools supports side-by-side review, painting of previously-viewed areas, percent coverage tracking, and multiple screen layouts including forward, reverse, fisheye and flat volume rendered views. Tools for contrast subtraction, “Cube View”, and integrated contextual reporting may be supported. Display and management of candidate markers from optional detection engines may be supported, including iNtuition's Spherefinder.

The Volumetric Histogram tools allow a volume of interest to be segmented and analyzed for composition. Research applications include the analysis of low-attenuation regions of the lungs, threshold-based division of tumors into voxel populations, investigation of thrombosed vessels or aneurysms, or other pathology.

Findings workflow tools provide a framework for tracking findings across serial examinations. A database holds measurements and key images, and provides support for structured comparisons and tabulated reporting of findings over time, such as the RECIST 1.1 approach for presenting serial comparisons. The Annotation and Image Markup (AIM) XML schema may be supported, for automated integration with voice-recognition systems or clinical databases, and Word-based reports may be derived from the database.

With these tools, any two CT, PET, MR or SPECT series, or any two-series combination thereof can be overlaid with one assigned a semi-transparent color coding and the other shown in grayscale and volume rendering for anatomical reference. Automatic registration is provided and subtraction to a temporary series or to a saved, third series is possible. Support for PET/MR visualization is included.

Certain MR examinations (for example, Breast MR) involve a series of image acquisitions taken over a period of time, where certain structures become enhanced over time relative to other structures. These tools feature the ability to subtract a pre-enhancement image from all post-enhancement images to emphasize visualization of enhancing structures (for example, vascular structures and other enhancing tissue). Time-dependent region-of-interest tools may be provided to plot time-intensity graphs of a given region.

Parametric mapping tools are an enhancement to the Multi-Phase MR tools, the parametric mapping option pre-calculates overlay maps where each pixel in an image is color-coded depending on the time-dependent behavior of the pixel intensity. As an example, this tool can be used in Breast MR to speed identification and investigation of enhancing regions.

The MultiKv tools provide support for Dual Energy and Spectral Imaging acquisitions from multiple vendors, providing standard image processing algorithms such as segmentation or contrast suppression, as well as generic toolkits for precise analysis and development of new techniques.

These examples, and most functions of current advanced image analyses and clinical data analyses are capable of being supported in the Peer Review Platform. However, the capabilities of engines as well as engines of engines goes much further and can accommodate tools with higher intelligence and automation, as well as to deliver individually tailored workflows by adapting engines to the individual or group preferences.

The embodiments described above can be applied to a variety of medical areas. For example, the techniques described above can be applied to vessel analysis (including Endovascular Aortic Repair (EVAR) and electrophysiology (EP) planning). Such vessel analysis is performed for interpretation of both coronary and general vessel analysis such as carotid and renal arteries, in addition to aortic endograft and electro-physiology planning. Tools provided as cloud services of the platform for locally-sited or cloud sited deployment include auto-centerline extraction, straightened view, diameter and length measurements, color overlays, fusion mapping, Curved Planar Reformation (CPR) and axial renderings, as well as charting of the vessel diameter vs. distance and cross-sectional views. The vessel track tool provides a Maximum Intensity Projection (MW) view in two orthogonal planes that travels along and rotates about the vessel centerline for ease of navigation and deep interrogation. Plaque analysis tools provide detailed delineation of non-luminal structure such as soft plaque, calcified plaque and intra-mural lesions.

In addition, the techniques described above can be utilized in the area of endovascular aortic repair. According to some embodiments, vascular analysis tools provided as similar cloud services support definition of report templates which captures measurements for endograft sizing. Multiple centerlines can be extracted to allow for planning of EVAR procedures with multiple access points. Diameters perpendicular to the vessel may be measured along with distances along the two aorto-iliac paths. Custom workflow templates may be used to enable the major aortic endograft manufactures' measurement specifications to be made as required for stent sizing. Sac segmentation and volume determination with a “clock-face” overlay to aid with documenting the orientation and location of branch vessels for fenestrated and branch device planning, may also be used. Reports containing required measurements and data may be generated.

The techniques described above can also be applied in the left atrium analysis mode, in which semi-automated left atrium segmentation of each pulmonary vein ostium is supported with a distance pair tool, provided as cloud services, for assessment of the major and minor vein diameter. Measurements are automatically detected and captured into the integrated reporting system. These capabilities can be combined with other vessel analysis tools to provide a comprehensive and customized EP planning workflow for ablation and lead approach planning.

The techniques described above can also be utilized in calcium scoring. Identification of coronary calcium is supported with Agatston, volume and mineral mass algorithms being totaled and reported. Results may be stored in an open-format database along with various other data relating to the patient and their cardiovascular history and risk factors. A customized report can be automatically generated based upon these data. Also includes report generation as defined by the Society of Cardiovascular Computed Tomography (SCCT) guidelines.

The techniques described above can also be utilized in a time-volume analysis (TVA), which may include fully-automated calculation of left ventricular volume, ejection fraction, myocardial volume (mass) and wall thickening from multi-phasic data.

The techniques described above can also be utilized in the area of segmentation analysis and tracking (SAT), which includes supports analysis and characterization of masses and structures in various scans, including pulmonary CT examinations. Features include segmentation of masses, reporting of dimensions and volume, graphical 3D display of selected regions, support for follow-up comparisons including percent volume change and doubling time, and support for application and review of filter results (e.g., sphericity).

The techniques described above can also be utilized in the area of fly through which may include features of automatic segmentation and centerline extraction of the colon, 2D review includes side-by-side synchronized supine and prone data sets in either axial, coronal or sagittal views with representative synchronized endoluminal views. 3D review includes axial, coronal and sagittal MPR or MIP image display with large endoluminal view and an unfolded view that displays the entire colon. Coverage tracking is supported to ensure 100% coverage with stepwise review of unviewed sections, polyp identification, bookmark and merge findings, as well as a cube view for isolating a volume of interest and an integrated contextual reporting tool. Support is provided for use of filter results (e.g., sphericity).

The techniques described above can also be utilized in the area of time-dependent analysis (TDA), which provides assessment analysis of the time-dependent behavior of appropriate computerized tomographic angiography (CTA) and/or MRI examinations, such as within cerebral perfusion studies. Multiple time-dependent series are analyzed at the same time, and a procedural workflow for selecting input and output function and regions of interest is provided. Exportation of values for blood flow, blood volume and transit time maps are supported or exported in DICOM or other image formats. Other outputs include calculation of various time-dependent parameters.

The techniques described above can also be utilized in the area of CTA-CT subtraction, which includes automatic registration of pre- and post-contrast images, followed by subtraction or dense-voxel masking technique which removes high-intensity structures (like bone and surgical clips) from the CTA scan without increasing noise, and leaving contrast-enhanced vascular structures intact.

The techniques described above can also be utilized in dental analysis, which provides a CPR tool which can be applied for review of dental CT scans, offering the ability to generate “panoramic” projections in various planes and of various thicknesses, and cross-sectional MPR views at set increments along the defined curve plane.

The techniques described above can also be utilized in the area of multi-phase MR (basic, e.g. breast, prostate MR). Certain MR examinations (for example, breast, prostate MR) involve a series of image acquisitions taken over a period of time, where certain structures become enhanced over time relative to other structures. Function include the ability to subtract a pre-enhancement image from all post-enhancement images to emphasize visualization of enhancing structures (for example, vascular structures and other enhancing tissue). Time-dependent region-of-interest tools are provided to plot time-intensity graphs of a given region.

The techniques described above can also be utilized in parametric mapping (e.g. for multi-phase Breast MR), in which the parametric mapping module pre-calculates overlay maps where each pixel in an image is color-coded depending on the time-dependent behavior of the pixel intensity.

The techniques described above can also be utilized in finding sphericity in objects within image datasets. This is often used with lung or colon CT scans to identify potential areas of interest.

The techniques described can also be utilized in fusion for CT/MR/PET/SPECT. Any two CT, PET, MR or SPECT series, or any two-series combination can be overlaid with one assigned a semi-transparent color coding and the other shown in grayscale and volume rendering for anatomical reference. Automatic registration is provided and subtraction to a temporary series or to a saved, third series is possible.

The techniques described above can also be utilized in the area of Lobular Decomposition. Lobular Decomposition is an analysis and segmentation tool that is designed to detect and segment anatomical structures. For any structure or organ region which is intertwined with a tree-like structure (such as an arterial and/or venous tree), the tool calculates volumes of interest, as well as the trees related to it, and partitions the volumes into lobes or territories which are most proximal to the tree or any specific sub-branch thereof. This generic and flexible tool has potential research applications in analysis of the liver, lung, heart and various other organs and pathological structures.

The techniques described above can also be utilized in the area of volumetric histogram calculations. Volumetric histogram partitions a given volume of interest based on constituent voxels creating groups or populations of different intensity or density ranges. This can be used, for example, to support research into disease processes such as cancer (where it is desirable to analyze the composition of tumors, in an attempt to understand the balance between active tumor, necrotic tissue, and edema), or emphysema (where the population of low-attenuation voxels in a lung CT examination may be a meaningful indicator of early disease).

The techniques described above can also be utilized in the area of motion analytics. Motion analytics provides a powerful 2D representation of a 4D process, for more effective communication of findings when interactive 3D or 4D display is not available. Any dynamic volume acquisition, such as a beating heart, can be subjected to the motion analysis, to generate a color-coded “trail” of outlines of key boundaries, throughout the dynamic sequence, allowing a single 2D frame to capture and illustrate the motion, in a manner that can be readily reported in literature. The uniformity of the color pattern, or lack thereof, reflects the extent to which motion is harmonic, providing immediate visual feedback from a single image.

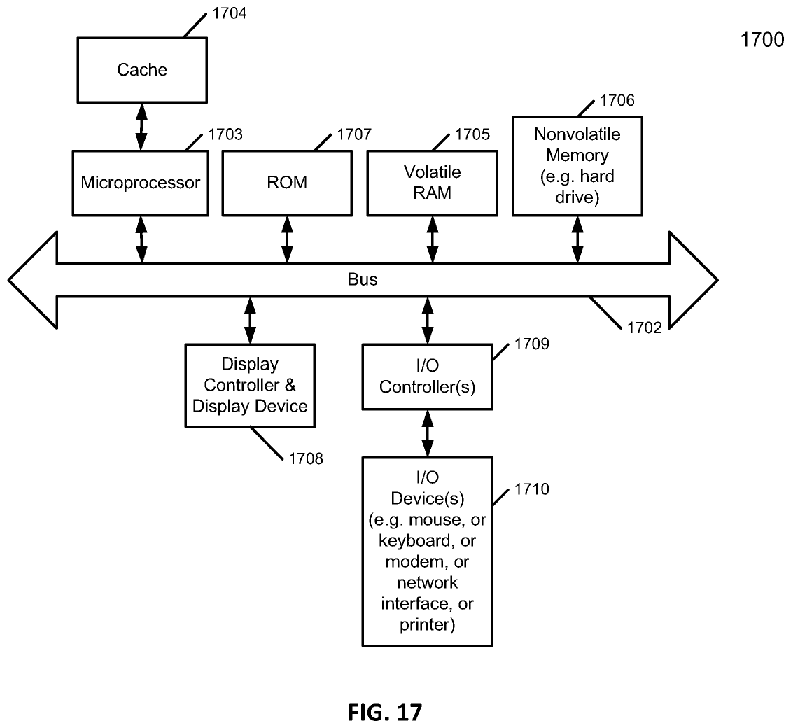

FIG. 17 is a block diagram of a data processing system, which may be used with one embodiment of the invention. For example, the system 1700 may be used as part of a server or a client as described above. For example, system 1700 may represent image processing server 110 described above, which is communicatively coupled to a remote client device or another server via network interface 1710. Note that while FIG. 17 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used with the present invention.

As shown in FIG. 17, the computer system 1700, which is a form of a data processing system, includes a bus or interconnect 1702 which is coupled to one or more microprocessors 1703 and a ROM 1707, a volatile RAM 1705, and a non-volatile memory 1706. The microprocessor 1703 is coupled to cache memory 1704. The bus 1702 interconnects these various components together and also interconnects these components 1703, 1707, 1705, and 1706 to a display controller and display device 1708, as well as to input/output (I/O) devices 1710, which may be mice, keyboards, modems, network interfaces, printers, and other devices which are well-known in the art.

Typically, the input/output devices 1710 are coupled to the system through input/output controllers 1709. The volatile RAM 1705 is typically implemented as dynamic RAM (DRAM) which requires power continuously in order to refresh or maintain the data in the memory. The non-volatile memory 1706 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 15 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 1702 may include one or more buses connected to each other through various bridges, controllers, and/or adapters, as is well-known in the art. In one embodiment, the I/0 controller 1709 includes a USB (Universal Serial Bus) adapter for controlling USB peripherals. Alternatively, I/0 controller 1709 may include an IEEE-1394 adapter, also known as FireWire adapter, for controlling FireWire devices.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the preceding figures may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

In the foregoing specification, embodiments of the invention have been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

1. A system for review of medical images, comprising:
a primary reviewer system;
an image processing server;
a peer reviewer system;
wherein the primary reviewer system is configured to receive a plurality of sets of medical images and coordinate review of the plurality of sets of medical images by a first set of humans for findings in the medical images;
the image processing server configured to receive the plurality of sets of medical images and a first set of evaluation results indicating findings identified in plurality of sets of medical images by the first set of humans;
wherein the image processing server is configured to identify findings in the plurality of sets of medical images using one or more image processing engines and produce a second set of evaluation results indicating findings identified in plurality of sets of medical images by the one or more processing engines;
wherein the image processing server is configured to compare the first set of evaluation results to the second set of evaluation results for the plurality of sets of medical images; and
wherein the image processing server is configured to select a set of medical images of the plurality of sets of medical images for peer review in response to a discrepancy between the first set of evaluation results and the second set of evaluation result for the set of medical images;
wherein the image processing server is configured to provide the selected set of medical images to the peer reviewer system;
wherein the peer reviewer system is configured to coordinate review of the sets of medical images received from the image processing server by a second set of humans for findings in the medical images and produce a third set of evaluation results indicating findings identified in plurality of sets of medical images by the second set of humans.

2. The system of claim 1, further comprising a logic circuit configured to receive the first set of evaluation results, the second set of evaluation results and the third set of evaluation results;
wherein the logic circuit is configured to invalidate operations of the one or more image processing engines in response to identifying identify inconsistencies between the first set of evaluation results, the second set of evaluation results, and the third set of evaluation results.

3. The system of claim 1, further comprising a logic circuit configured to receive the first set of evaluation results, the second set of evaluation results and the third set of evaluation results;
wherein the logic circuit is configured to train the one or more image processing engines based on adjudicated results of the first set of evaluation results, the second set of evaluation results and the third set of evaluation results using a machine learning algorithm.

4. The system of claim 1, wherein the image processing server is configured to transmit an alert message in response to the comparison of the first set of evaluation results and the second set of evaluation results indicating a discrepancy.

5. The system of claim 1, further comprising:
receiving a third set of evaluation results from the peer reviewer system;
the third set of evaluation results indicating finding in the set of medical images identified by the second human;
using the third set of evaluation results to perform machine learning training of the one or more image processing engines to improve identification of findings by the one or more image processing engines.

6. The system of claim 1, wherein the one or more image processing engines include a plurality of image processing engines configured to process the medical images according to a predetermined order, wherein each image processing engine of the plurality is a discrete automated image processing engine that is launched and executed by a processor independent of all other image processing engines in the plurality of image processing engines.

7. The system of claim 1, further comprising:
comparing the first set of evaluation results against the second set of evaluation results; and
transmitting an alert to a predetermined device, in response to determining that the first set of evaluation results is inconsistent with the second set of evaluation results.

8. The system of claim 1, wherein performing machine learning training includes using a machine-learning algorithm based on the first result and the second result to train a supervisory engine of the plurality of image processing engines.

9. The system of claim 1, wherein the one or more image processing engines include a plurality of image processing engines;
further comprising tracking statistics of operations of the plurality of image processing engines, including data indicating which image processing engine performs operations on which clinical study.

10. The system of claim 1, wherein the one or more image processing engines include a plurality of image processing engines;
wherein the plurality of image processing engines are configured to process different portions of the medical images concurrently in a distributed manner, and wherein a supervisor engine allocates and assigns tasks to remaining processing engines of the plurality.

11. The system of claim 1, wherein the one or more image processing engines include a plurality of image processing engines;
further comprising using a supervisory engine to train at least two image processing engines of the plurality of images processing engines, each of the at least two image processing engines having a separate weighting, the weighting being used to give weight to findings of each of the at least two image processing engines when determining the first result, the first result including discovered findings.

12. The system of claim 1, wherein the plurality of sets of medical images are associated with a patient.

13. The system of claim 1, wherein the plurality of sets of medical images are associated with a clinical study.

14. The system of claim 1, wherein the second set of evaluation results indicate a severity of the findings identified in plurality of sets of medical images by the one or more processing engines.

15. The system of claim 1, wherein the second set of evaluation results indicate a risk of the findings identified in plurality of sets of medical images by the one or more processing engines.

16. A computer-implemented method for evaluation of medical images, the method comprising:
retrieving a set of medical images and a first set of evaluation results from a medical data source;
wherein the first set of evaluation results indicate abnormal findings identified in the set of medical images by a first human;
processing the retrieved medical images using one or more image processing engines configured to detect abnormal findings in the medical images to produce a second set of evaluation results;
comparing the first set of evaluation results to the second set of evaluation results; and
providing the set of medical images to a peer reviewer system for review of the set of images results by a second human in response to either:
a discrepancy between the first set of evaluation results to the second set of evaluation result, or
the first set of evaluation results and the second set of evaluation result both indicating an abnormal finding in the set of medical images.

17. The method of claim 16, further comprising:
receiving a third set of evaluation results from the peer reviewer system;
the third set of evaluation results indicating abnormal finding in the set of medical images identified by the second human;
using the third set of evaluation results to perform machine learning training of the one or more image processing engines to improve identification of abnormal findings by the one or more image processing engines.

18. The method of claim 16, wherein the one or more image processing engines include a plurality of image processing engines configured to process the medical images according to a predetermined order, wherein each image processing engine of the plurality is a discrete automated image processing engine that is launched and executed by a processor independent of all other image processing engines in the plurality of image processing engines.

19. The method of claim 16, further comprising:
comparing the first set of evaluation results against the second set of evaluation results; and
transmitting an alert to a predetermined device, in response to determining that the first set of evaluation results is inconsistent with the second set of evaluation results.

20. The method of claim 16, wherein performing machine learning training includes using a machine-learning algorithm based on the first result and the second result to train a supervisory engine of the plurality of image processing engines.

21. The method of claim 16, wherein the one or more image processing engines include a plurality of image processing engines;
further comprising tracking statistics of operations of the plurality of image processing engines, including data indicating which image processing engine performs operations on which clinical study.

22. The method of claim 16, wherein the one or more image processing engines include a plurality of image processing engines;
wherein the plurality of image processing engines are configured to process different portions of the medical images concurrently in a distributed manner, and wherein a supervisor engine allocates and assigns tasks to remaining processing engines of the plurality.

23. The method of claim 16, wherein the one or more image processing engines include a plurality of image processing engines;
further comprising using a supervisory engine to train at least two image processing engines of the plurality of images processing engines, each of the at least two image processing engines having a separate weighting, the weighting being used to give weight to findings of each of the at least two image processing engines when determining the first result, the first result including discovered abnormal findings.

24. The method of claim 16, wherein the set of medical images are associated with a patient.

25. The method of claim 16, wherein the set of medical images are associated with a clinical study.

26. A system for review of medical data, comprising:
a primary reviewer system;
an data processing server;
a peer reviewer system;
wherein the primary reviewer system is configured to receive a plurality of sets of medical data and coordinate review of the plurality of sets of medical data by a first set of humans for findings in the medical data;
the data processing server configured to receive the plurality of sets of medical data and a first set of evaluation results indicating findings identified in plurality of sets of medical data by the first set of humans;
wherein the data processing server is configured to identify findings in the plurality of sets of medical data using one or more data processing engines and produce a second set of evaluation results indicating findings identified in plurality of sets of medical data by the one or more processing engines;
wherein the data processing server is configured to compare the first set of evaluation results to the second set of evaluation results for the plurality of sets of medical data; and
wherein the data processing server is configured to select a set of medical data of the plurality of sets of medical data for peer review in response to a discrepancy between the first set of evaluation results and the second set of evaluation result for the set of medical data;
wherein the data processing server is configured to provide the selected set of medical data to the peer reviewer system;
wherein the peer reviewer system is configured to coordinate review of the sets of medical data received from the data processing server by a second set of humans for findings in the medical data and produce a third set of evaluation results indicating findings identified in plurality of sets of medical data by the second set of humans.

27. The system of claim 26, wherein the plurality of set of medical data include a combination of two or more types of data selected or a set of data types including medical image data in a DICOM format, medical image data in a non-DICOM format, scheduling data, registration data, demographic data, prescription data, billing data, insurance data, dictation data, report data, workflow data, EKG data, best practices reference materials, reference materials, and training materials.
